(19) United States
(12) Patent Application Publication
(10) Pub. No.: US 2003/0124629 A1
(43) Pub. Date: Jul. 3, 2003

(54) Methods of detecting a cell

(76) Inventors: Eric Tse, Cambridge (GB); Terence Rabbitts, Cambridge (GB)

Correspondence Address: PALMER & DODGE, LLP, KATHLEEN M. WILLIAMS, 111 HUNTINGTON AVENUE, BOSTON, MA, 02199, US

(21) Appl. No.: 10/265,002

(22) Filed: Oct. 4, 2002

Related U.S. Application Data
(63) Continuation-in-part of application No. PCT/GB01/01540, filed on Apr. 4, 2001.

(30) Foreign Application Priority Data
Apr. 4, 2000 (GB) 0008256.0
Apr. 4, 2002 (GB) 0008254.5

(51) Int. Cl. G01N 33/574

(52) U.S. Cl. 435/7.23

(57) ABSTRACT

We describe a method of inducing a cell to generate a detectable signal. The method comprises the steps of providing a cell comprising an entity and providing a first reporter and a second reporter, in which a stable interaction of the first reporter with the second reporter leads to generation of a detectable signal. The first reporter and the second reporter are allowed to bind to the entity, such that binding of the reporters to the entity leads to stable interaction of the first reporter with the second reporter and generation of a signal. The signal is preferably the activation of a cell killing mechanism.

### RELATED APPLICATIONS

This application is a Continuation of PCT/GB01/01540 filed Apr. 4, 2001, which claims priority to GB Patent Application No. 0008256.0 filed Apr. 4, 2000 and GB Patent Application No. 0008254.5 filed Apr. 4, 2000.

### FIELD OF THE INVENTION

This invention relates to methods of detecting proteins and other entities within a cell, in particular, an entity which is associated with an abnormal cell.

### BACKGROUND OF THE INVENTION

Cancer is characterised by genetic abnormalities that affect growth control, differentiation and survival of cells (Vogelstein, B. & Kinzler, K. W. The Genetic Basis of Human Cancer (McGraw-Hill, 1998)). Mutations in oncogenes and chromosomal translocations, which give rise to enforced expression of oncogenes or chimaeric fusion proteins, are frequently observed in tumours (Rabbitts, T. H. 1994, Nature 372, 143-149). The protein products of these abnormal genes are unique to cancer cells and are therefore tumour specific antigens. Elimination of the mutant proteins or inhibition of their functions has been shown to be effective in controlling cancer growth and progression (Chin et al. (1999), Nature 400, 468-472; Felsher, et al., (1999) Mol Cell 4, 199-207; Huettner, et al. (2000), Nat Genet 24, 57-60). However, although such proteins are potential targets for therapeutic intervention, they are mainly intracellular proteins and therefore present practical difficulties in the design of therapeutic strategies.

One approach has been the intracellular expression of antibodies or antibody fragments to inactivate mutant proteins, either by neutralising their functions (Cochet, et al. (1998), Cancer Res 58, 1170-6) or by preventing them from reaching the necessary cell compartments where they exert their effects (Wright et al., (1997) Gene Ther 4, 317-22). However, it is not always possible to predict in advance whether an antibody will bind to a particular protein, particularly within the internal environment of a cell. Selection methods which directly identify antibodies capable of binding intracellularly to antigens have been proposed, such as an in vivo two-hybrid system for selecting antibodies with binding capability inside mammalian cells. Such a method is described in our earlier United Kingdom application number 9905510.5 and International Patent Application number PCT/GB00/00876, hereby incorporated by reference.

However, even if a candidate antibody is identified which is likely to bind in vivo, there is no guarantee that this antibody will be able to neutralise the functions of the tumour protein or prevent its intracellular localisation. Furthermore, the tumour phenotype may be caused by mutation at more than one protein, so that neutralising the function of one of the mutant proteins will not necessarily be effective in halting tumour cell proliferation. There is therefore a need for a more effective way of killing tumour cells using antibodies.

As tumour associated antigens are characteristic of cancerous cells, detection of these antigens may be used as a means of diagnosis of cancer in a patient. Expression of antigen may also be detected as a means of genotyping an individual. Furthermore, detection of specific markers may be used as a means of tissue typing. In all these cases, presence of antigen may be determined by exposing a labelled antibody to suspect cells and detecting antibody-antigen binding. This method is however only suitable for detection of antigens which are expressed extracellularly. There is therefore a need for an effective method of detecting intracellular markers.

### SUMMARY OF THE INVENTION

We have now found that instead of using intracellular tumour specific antigens as direct targets for a neutralising antibody, it is possible to detect a cell by the use of a pair of reporters which bind to an entity within the cell and co-operate to generate a detectable signal. This signal may be used to identify the cell. When the cell detected is a tumour cell or other diseased cell, the signal may advantageously be the activation of a cell-killing mechanism, and the tumour or diseased cell may be eliminated this way.

Accordingly, we provide in one aspect of the invention, a method of inducing a cell to generate a detectable signal, the method comprising the steps of: (a) providing a cell comprising an entity; (b) providing a first reporter and a second reporter, in which a stable interaction of the first reporter with the second reporter leads to generation of a detectable signal; (c) allowing the first reporter and the second reporter to bind to the entity, such that binding of the reporters to the entity leads to stable interaction of the first reporter with the second reporter and generation of a signal.

We provide, according to a second alternative aspect of the invention, a method of detecting an entity within a cell, the method comprising the steps of: (a) providing a first reporter and a second reporter, in which a stable interaction of the first reporter with the second reporter leads to generation of a detectable signal; (b) allowing the first reporter and the second reporter to bind to the entity, such that binding of the reporters to the entity leads to stable interaction of the first reporter with the second reporter and generation of a signal; and (c) detecting the entity by monitoring the signal.

There is provided, according to a third and alternative aspect of the invention, a method of killing a cell, the method comprising the steps of: (a) providing a cell comprising an entity; (b) providing a first reporter comprising a first immunoglobulin and a second reporter comprising a second immunoglobulin; (c) providing a first caspase molecule linked to the first immunoglobulin and a second caspase molecule linked to the second immunoglobulin, the first and the second caspase molecules being capable of stably interacting to generate a caspase activity to cause apoptosis in the cell; and (d) allowing the first immunoglobulin and the second immunoglobulin to bind to the entity, such that binding of the reporters to the entity leads to stable interaction of the caspase molecules to generate caspase activity and apoptosis in the cell.

By “entity” we mean any cellular component. The entity is preferably a peptide, polypeptide, protein, genomic DNA, messenger RNA, transfer RNA, a subcellular structure or an intracellular pathogen. Nascent polypeptides and intracellular polypeptide precursors are also included within the term “entity”. Preferably, the entity is a polypeptide associated with a predetermined condition of the cell or an organism from which the cell is derived.

The cell may be a differentiated cell or an abnormal cell such as a tumour cell, a diseased cell or an infected cell. By “abnormal cell”, we mean a cell which is diseased, infected or which otherwise does not exhibit the characteristics or behaviour of a normal cell. The entity may therefore be a cellular component associated with Alzheimer's disease or Down's Syndrome, for example, a neurofibrillary tangle or a senile plaque. The entity may also be a mutant beta amyloid precursor protein or a mutant ubiquitin-B protein. It is known that frameshift mutations in a RNA encoding beta amyloid precursor protein or ubiquitin-B protein are associated with Alzheimer's Disease and Down's Syndrome, and accordingly, the entity of the invention may be a frameshifted RNA encoding such a protein. The entity may also be an infectious form of the prion protein (PrPSc), which is associated with mammalian spongiform encephalopathies such as Creutzfeld-Jacob Disease (CJD), new variant CJD, or Bovine Spongiform Encephalopathy (BSE or Mad Cow Disease). The entity may also be a protein or other molecule associated with AIDS (acquired immunodeficiency syndrome) or an autoimmune disease.

Preferably, the entity is a cancer associated- or tumour associated protein or a disease specific protein. Most preferably, the entity is a oncogenic protein resulting from a mutation in the cell or a progenitor of the cell. The mutant oncogenic protein may be p21 ras. Preferably, one of the first reporter and second reporter binds to a target present in the mutant oncogenic protein but not in a corresponding wild type protein, and the other of the first reporter and second reporter binds to a target present in both the mutant oncogenic protein and the wild-type protein.

Alternatively, the mutant oncogenic protein is a chimaeric fusion protein resulting from a chromosomal translocation. An example of a chromosomal translocation is the Philadelphia translocation which gives rise to the BCR-ABL fusion protein. Preferably, one of the first reporter and the second reporter binds to a target comprising an SH2 domain, and the other of the first reporter and the second reporter binds to a target comprising an SH2-binding site.

The mutant oncogenic protein may also be a mutated p53 protein; preferably the p53 protein is a tetramer made of p53 subunits. Preferably, the first reporter and the second reporter bind to identical targets present in the mutated p53 protein.

In general, where the entity is a natural cellular component which multimerises, the first and second molecule may be identical and may be brought into association through the multimerisation of the entity. Where the entity is a mutant entity, the ability to multimerise is preferably retained. Preferably, one or both of the first and second molecules, or an additional third molecule, may have a tethering activity in order to improve the cooperation of the reporter groups by bringing them closer together in the multimerised entity.

A “signal”, as used here, is any detectable event, preferably the activation of a cell-killing mechanism. In a preferred embodiment of the invention the cell-killing mechanism is apoptosis or programmed cell death. Preferably, the first reporter and the second reporter each comprise a caspase molecule, and the binding of the reporters to their targets leads to auto-activation of the caspase molecules to initiate apoptosis. Alternatively, the signal may be the generation of an enzymatic activity, such as protease activity, transcriptional activity or luminescence inducing activity.

“Stable interaction” may be defined as an interaction which permits functional cooperation of the first and second reporters in order to give rise to a detectable signal. Preferably, the first reporter and/or the second reporter comprise polypeptides which associate to form a molecule which is capable of generating a signal.

By “protease activity”, we mean the activity of a protease or any other enzyme which is capable of cleaving a protein or peptide. Protease activity may be assayed by monitoring degradation of a suitable-substrate, by methods known in the art. Preferably, the polypeptide substrate consists of or comprises the entity which is detected. The protease activity may be a cysteine protease activity, for example, caspase activity.

In one embodiment of the invention, the protease activity comprises a caspase activity. Preferably, the caspase activity is generated by the stable interaction of the first reporter with the second reporter. Thus, in this embodiment, stable interaction between the first reporter and the second reporter directly generates protease activity. Preferably, the caspase is caspase 3 or caspase 8. More preferably the first reporter and the second reporter each comprise a caspase molecule. Even more preferably, binding of the reporters to their targets leads to auto-activation of the caspase molecules and activation of apoptosis in the cell. Most preferably, the first reporter and the second reporter each comprise caspase 3. Caspases are a group of proteins which are involved in triggering apoptosis, and the term “caspase” is known in the art. Examples of caspases are those described in Takahashi, Int J Hematol December 1999;70(4):226-32.

In a further embodiment, the protease activity may be associated with a proteasome which destroys proteins as part of ubiquitin-mediated proteolysis. Preferably, the proteasome is a 26S proteasome. The first and second reporters may comprise domains of a component involved in a ubiquitin-mediated proteolysis pathway, preferably domains of a F-box protein. Reconstitution of the component (e.g., F-box) by stable interaction of the first and second reporters by binding to the entity to be detected leads to ubiquitin labelling of the entity or a polypeptide comprising that entity. The entity and/or polypeptide is marked for destruction and subsequently destroyed by a proteasome (preferably the 26S proteasome), and the signal may be detected by means known in the art. It will be appreciated that in this embodiment, association of the first and second reporters does not itself generate protease activity directly, but that protease activity is generated indirectly by such association. It will further be appreciated that where the polypeptide that is destroyed is essential for cell viability, such as a metabolic enzyme, a transcription factor, a structural protein such as a membrane protein, etc, cell death (which is itself detectable) may result. The same situation arises where destruction of the polypeptide otherwise leads to cell death, for example, by triggering apoptosis.

The enzymatic activity may be a transcriptional activity, and the first reporter and the second reporter may preferably comprise domains of a transcription factor. By “transcriptional activity”, we mean activity which induces production of messenger RNA from a transcription unit. For example, transcriptional activity is demonstrated by a transcription factor or any other regulatory molecule which modulates gene expression within a cell. Preferably, either the first reporter or the second reporter comprises the DNA binding domain (DBD) of Gal4, and the other of the first reporter and the second reporter comprises a VP16 activation domain. The signal may be detected by monitoring the expression of a reporter gene. The reporter gene may encode an enzyme capable of catalysing an enzymatic reaction with a detectable end-point. Alternatively, the reporter gene may encode a molecule capable of regulating cell growth, such as providing a required nutrient. Preferably, the reporter gene encodes Green Fluorescent Protein (GFP), luciferase, β-galactosidase, or chloramphenicol acetyl transferase (CAT). Most preferably, the reporter gene is CD4.

The invention moreover comprises the use of transcriptional regulation mechanisms as reporter systems. For example, transcriptional regulation of diphtheria toxin expression may be dependent on the cooperation of the first and second reporters. Thus a first reporter may comprise a coding sequence encoding a toxin under the control of a minimal promoter, which has negligible background expression levels; a second reporter may regulate the expression of a transcription factor, either endogenous or exogenous, which in turn upregulates toxin expression.

The enzymatic activity may be luminescence inducing activity. “Luminescence” refers to the production of light or other radiation by a chemical reaction, and includes bioluminescence or chemiluminescence. Preferably, the luminescence inducing activity is preferably provided by luciferase. More preferably, the first and second reporter comprise polypeptide domains of luciferase, which when brought together produce luciferase activity.

The signal may be emission or absorption of electromagnetic radiation, for example, light. Preferably, the signal is a fluorescent signal. More preferably, the fluorescent signal is emitted from a fluorescent chemical or a fluorescent protein. Preferred fluorescent chemicals are fluorescein isothiocyanate and rhodamine, and preferred fluorescent proteins are Green Fluorescent Protein, Blue Fluorescent Protein, Cyan Fluorescent Protein, Yellow Fluorescent Protein and Red Fluorescent Protein. Most preferably, the fluorescent signal is modulated by fluorescent resonance energy transfer (FRET). The fluorescent signal is preferably detected by means of a fluorescence activated cell sorter (FACS).

The first reporter and the second reporter may bind to the same target site. Where this is so, the first reporter and the second reporter may bind to the same target site substantially simultaneously, or sequentially. Alternatively and preferably, the first reporter and the second reporter bind to different target sites. Where the reporters bind to different target sites, the target sites are advantageously adjacent, such that the reporters may cooperate functionally in accordance with the present invention.

Preferably, the first reporter comprises a first association means and the second reporter comprises a second association means, and the first and second reporters interact via their respective association means. More preferably, the first reporter comprises first binding means which binds to a first target on the entity, and the second reporter comprises second binding means which binds to a second target on the entity. Most preferably, the first reporter comprises the first association means linked to the first binding means, and the second reporter comprises the second association means linked to the second binding means.

The term “link” is intended to mean any form of covalent or non-covalent attachment between two moieties, for example the binding means and the association means. An example of a link is a peptide link in a fusion protein. Chemical coupling or conjugation between the moieties is expressly included. Furthermore, the binding means and the association means need not be linked directly, and linkage may occur via a linker peptide. The linker peptide may be a flexible or a structured linker peptide. Suitable flexible linker peptides may comprise one or more glycine residues, optionally in combination with other amino acid residues. A structured linker may comprise one or more proline residues, and may comprise a defined secondary structure.

By “binding means” we mean anything which is capable of specific binding to a target. Preferably, the binding means is a molecule; more preferably, the binding means is a polypeptide or protein.

Most preferably, the first binding means and/or the second binding means comprises a immunoglobulin, for example, an antibody or a T-cell receptor, or a fragment thereof. Preferably, the antibody is chosen from a Fv, a single chain Fv (scFv), a Fab or a F(ab′)₂. More preferably, the antibody is a single chain Fv. Most preferably, the antibody is an intracellular single chain Fv. Use of reporter(s) comprising immunoglobulins is preferred where the entity comprises protein or polypeptide. The entity may therefore comprise an epitope which is recognised by the immunoglobulin.

The term “immunoglobulin” refers to any member of the immunoglobulin superfamily, including T-cell receptors and antibodies, and includes any fragment of a natural immunoglobulin which is capable of binding to a target. A comprehensive review of immunoglobulins is provided in Male et al (1987), Advanced Immunology, J. B. Lippincott Company, Philadelphia. Included within the term “immunoglobulin” are intact immunoglobulins as well as antibody fragments such as Fv, a single chain Fv (scFv), a Fab or a F(ab′)₂. “Intracellular” as used here means inside a cell. An “intracellular antibody” is an antibody which is capable of binding to its target or cognate antigen within the environment of a cell, or in an environment which mimics an environment within the cell.

Where the association means and the binding means both comprise proteins, the reporter is preferably provided as a fusion protein comprising respective association means linked to binding means. Preferably, the first reporter and/or the second reporter are provided by expression of nucleic acid within the cell. Where the binding means comprises an antibody or a T-cell receptor, the nucleic acid is preferably obtained from a phage library encoding a repertoire of antibodies or T-cell receptors. A “repertoire” refers to a set of molecules generated by random, semi-random or directed variation of one or more template molecules, at the nucleic acid level, in order to provide a multiplicity of binding specificities.

Most preferably, the library is constructed from nucleic acids isolated from an organism which has been challenged with an antigen. Alternatively, the association means is linked to the binding means by chemical coupling.

Where the entity comprises a nucleic acid, the first reporter and/or the second reporter may bind to a target comprising a sequence located in the entity. Preferably, binding of the first and/or second reporter to the respective target occurs by means of nucleic acid hybridisation. More preferably, the first reporter and/or the second reporter comprises nucleic acid binding means capable of hybridising to a target sequence. Most preferably, the nucleic acid binding means comprises RNA or single stranded DNA.

The first and/or second reporters may interact via association means which comprise nucleic acid sequences. Stable interaction of the reporters may in this case be detected by monitoring absorbance of radiation, for example ultraviolet radiation. Alternatively, the nucleic acid association means may be linked to any components which are capable of generating the signals described here, and interaction between the reporters may be detected by monitoring the particular signal generated.

Where the association means and the binding means both comprise nucleic acid, the first and/or second reporter may be provided in the form of a contiguous nucleic acid sequence comprising the association means and the binding means.

Alternatively, the first and/or second reporter may be provided in the form of a hybrid comprising both nucleic acid and protein. Thus, the reporter may comprise a binding means comprising protein and an association means comprising nucleic acid, or conversely, a binding means comprising nucleic acid and an association means comprising protein. All that is important is that the binding of the binding means of the first reporter to its target brings the association means of the first reporter into stable association with the association means of the second reporter, when it is bound to its target, so that a signal is generated.

There is provided, according to a fourth alternative aspect of the invention, a pharmaceutical composition comprising an immunoglobulin-caspase fusion protein or conjugate, or a nucleic acid encoding an immunoglobulin-caspase fusion protein, together with a pharmaceutically acceptable carrier or diluent.

In a fifth alternative aspect of the invention, we provide an immunoglobulin-caspase fusion protein or conjugate, or a nucleic acid encoding an immunoglobulin-caspase fusion protein for use in a method of treatment or diagnosis of a cancer in a human or animal.

According to a sixth alternative aspect of the invention, there is provided these of an immunoglobulin-caspase fusion protein or conjugate, or a nucleic acid encoding an immunoglobulin-caspase fusion protein for the preparation of a medicament for the treatment or diagnosis of cancer in a human or animal.

In a seventh aspect of the present invention, there is provided a method of destruction of a polypeptide in a cell, the method comprising the steps of: (a) providing a cell comprising a polypeptide; (b) providing a first reporter and a second reporter, in which a stable interaction of the first reporter with the second reporter leads to generation of protease activity; and (c) allowing the first reporter and the second reporter to bind to the polypeptide, such that binding of the reporters to the polypeptide leads to stable interaction of the first reporter with the second reporter, generation of protease activity and proteolysis of the polypeptide.

According to an eighth aspect of the present invention, we provide a method of identifying the function of a gene, the method comprising the steps of: (a) providing a cell comprising a gene encoding a polypeptide; (b) providing a first reporter and a second reporter, in which a stable interaction of the first reporter with the second reporter leads to generation of protease activity; (c) allowing the first reporter and the second reporter to bind to the polypeptide, such that binding of the reporters to the polypeptide leads to stable interaction of the first reporter with the second reporter, generation of protease activity and proteolysis of the polypeptide; and (d) observing a phenotype.

A method according to this aspect is useful for functional genomics studies. Appropriate binding entities capable of targeting any protein entity may be constructed by following the detailed description provided here, to disrupt the function of the protein by its destruction. Functional “knock-outs” may therefore be created readily. The phenotype of the cell or animal or plant comprising the cell may then be observed to provide an indication of the function of the polypeptide or gene. Thus, for example, where a cell which has been targeted exhibits an arrested cell cycle phenotype, for example, it may be concluded that the protein in question which is targeted has a role in cell cycle function. The ability to target any protein by the use of suitably designed reporters, as described in further detail below, enables this application to have wide utility.

Preferably, the signal is generation of a protease activity associated with a proteasome, preferably a 26S proteasome. More preferably, the first reporter and the second reporter each comprise one or more domains of a F-box motif. Preferably, the binding of the reporters to the entity leads to ubiquitination of the entity, or a polypeptide comprising the entity. In a highly preferred embodiment of the invention, binding of the reporters to the entity leads to proteolysis of the entity, or a polypeptide comprising the entity. Such proteolysis preferably leads to death of the cell.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

### BRIEF DESCRIPTION OF THE DRAWINGS

FIG. 1. Mammalian Two-Hybrid ScFv-β-Galactosidase Transcription Assay

FIG. 1A is a diagram depicting the effect of co-transfecting expression vectors encoding the anti-β-galactosidase antibody fragment scFvR4-DBD fusion protein (DNA binding domain) and scFvR4-VP16 (VP16 transcriptional transactivating domain) fusion proteins in the CHO-CD4 reporter cell line. The scFvR4-DBD and scFvR4-VP16 fusion proteins, when bound to a β-galactosidase tetramer, can form a transcription complex which can bind to the chromosomal GAL4 DNA-binding site (DBS) which controls transcription of the CD4 reporter gene (Fearon et al., 1992, Proc. Natl. Acad. Sci. USA 89, 7958-7962). After this, CD4 protein molecules appear on the cell surface and can be assayed with the fluorescence activated cell sorter.

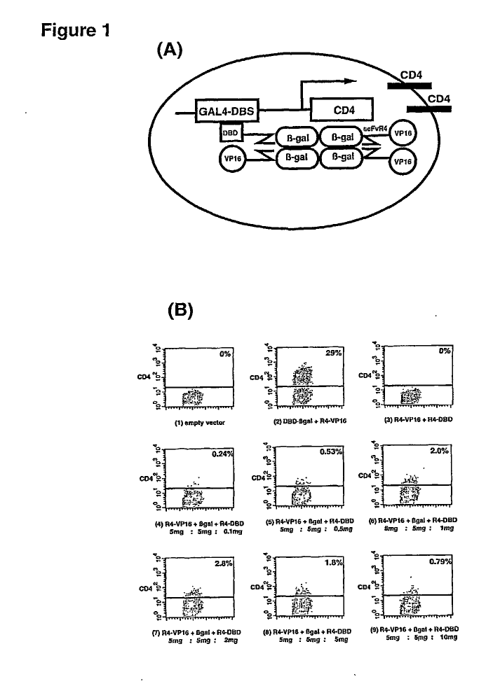

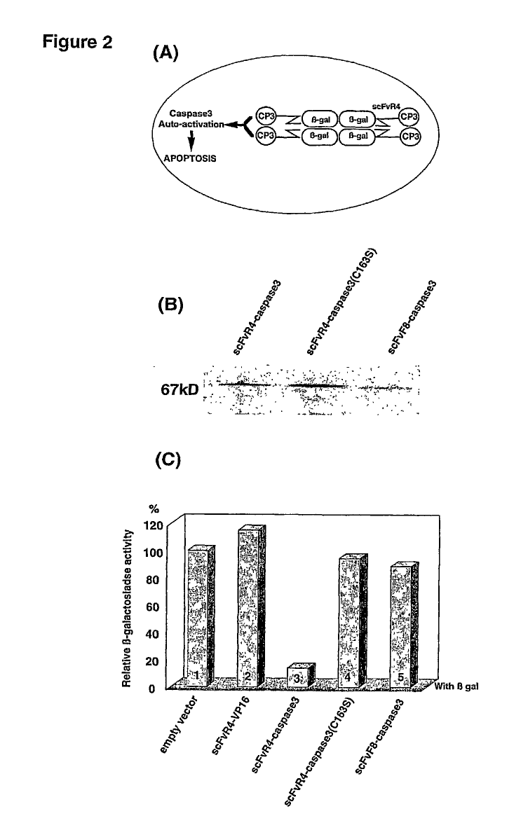

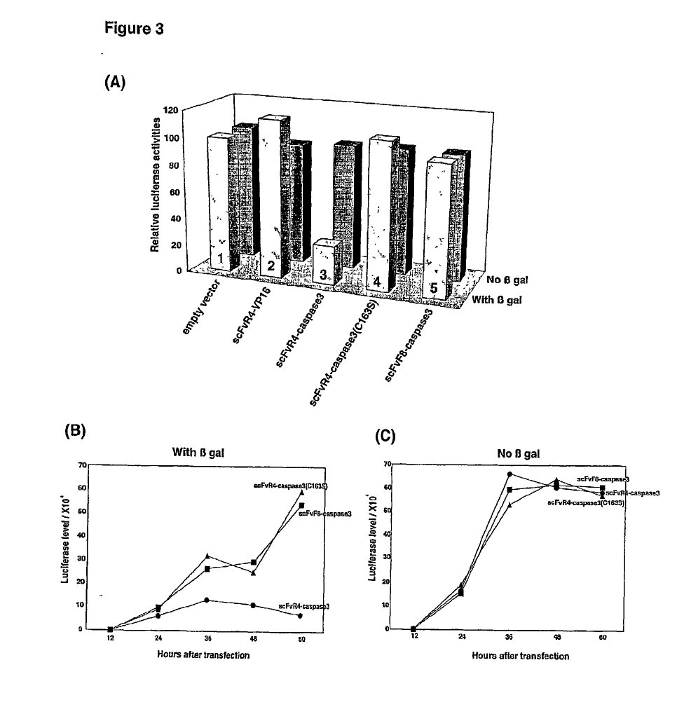

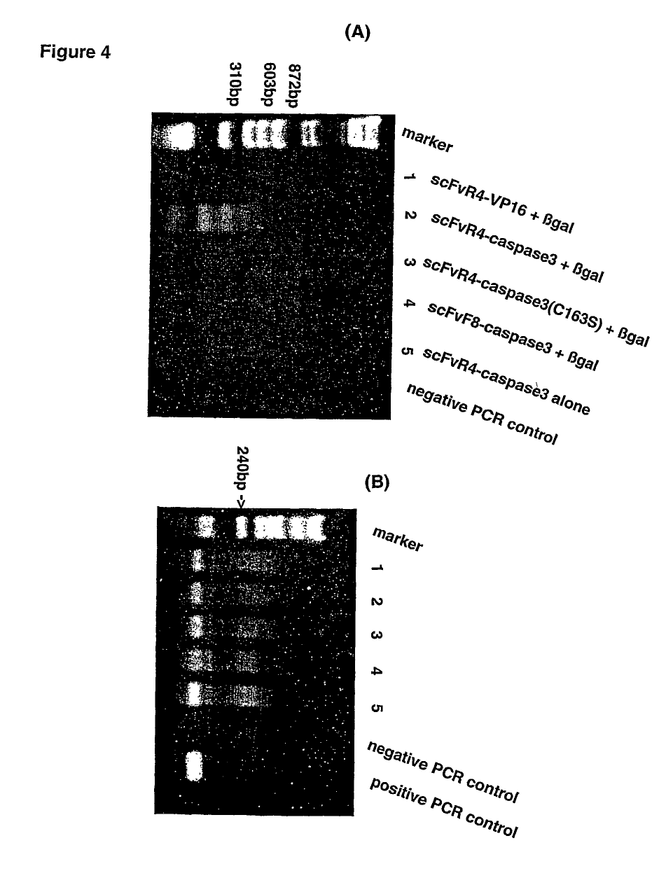

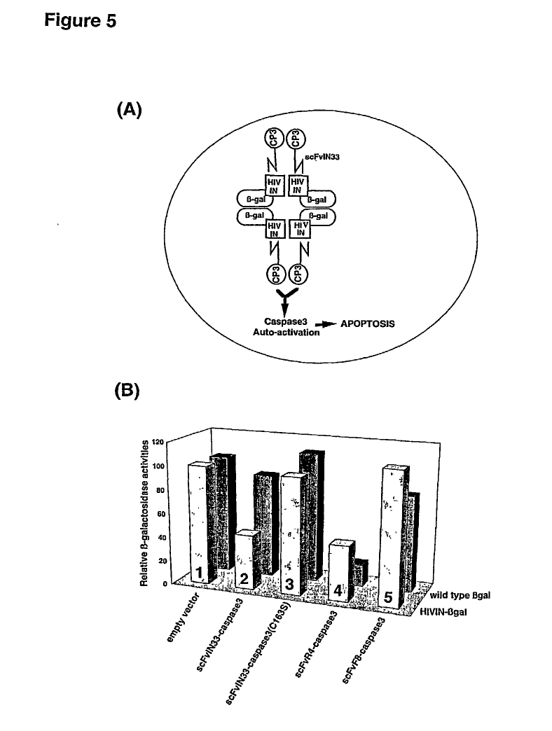

Results of such an experiment are shown in FIG. 1B. Panels A-I show FACs analyses of CHO-CD4 cells co-transfected with β-galactosidase expression clone pEF-βgal, together with various expression vectors. Panel 1: pEF-BOS vector only; Panel 2: DBD-βgal and scFvR4-VP16; Panel 3: scFvR4-VP16 and scFvR4-DBD; Panels 4-9: Variable amounts of scFvR4-VP16 and scFvR4-DBD as indicated. The amount of pEF-βgal plasmid (5 μg) is not varied.

DBD=GAL4 DNA binding domain; VP16=VP16 activation domain; DBS=DNA binding sites.

Induction of cell surface CD4 expression is assayed after 60 hours using anti-human CD4 antibody. The indicated percentage of CD4+cells after 48 hours is estimated using a FACSCalibur machine.

FIG. 2. Cell Death Mediated by Intracellular Antibody ScFvr4-Caspase3 Binding to β-Galactosidase

FIG. 2A is a diagram illustrating a model for possible binding of scFvR4-caspase3 fusion (scFv-CP3) to β-galactosidase (β-gal) tetramer causing dimerisation of caspase 3 and its auto-activation to trigger apoptosis.

FIG. 2B shows the results of Western analysis of scFv-fusion proteins expressed in CHO cells. CHO cells are transfected for 48 hours with scFvR4-caspase 3, scFvR4-caspase 3 (C163S) or scFvF8-caspase 3 expression vectors. Protein extracts are prepared, fractionated and transferred to nitrocellulose membranes. These are incubated with anti-caspase 3 antibody and secondary HRP conjugated anti-goat antibody. Detection is done by using ECL (enhanced chemiluminescence). Molecular weights are determined by co-electrophoresis of prestained protein molecular weight standards.

FIG. 2C shows the results of experiments in which CHO cells are transfected with plasmids expressing βgal (pEF-βgal) and the various scFv fusion proteins as indicated on the histogram. β-galactosidase levels are measured 60 hours after transfection and the data are the representative of two independent experiments, each carried out in duplicate. β-galactosidase levels are expressed relative to that obtained with pEF-βgal co-transfected with pEF-BOS vector alone, which was taken as 100%. Lane 1: empty vector; Lane 2: scFvR4-VP16; Lane 3: scFvR4-caspase3; Lane 4: scFvR4-caspase3(C163S); Lane 5: scFvF8-caspase3.

FIG. 3. Luciferase Activity in CHO Cells Co-Expressing ScFv-Fusions and β-Galactosidase

FIG. 3A shows results from experiments in which CHO cells are transfected with RSVluc, a luciferase expression plasmid (de Wet, et al., 1987, Mol Cell Biol 7, 725-37) together with the various scFv fusion expression plasmids in the presence (front row) or absence (back row) of the β-galactosidase expression vector, pEF-βgal, which serves to provide specific intracellular antigen. Luciferase levels are measured 60 hours after transfection. Levels are normalised to the level obtained for luciferase expression (100% value) when co-transfected with pEF-BOS vector only. Lane 1: empty vector; Lane 2: scFvR4-VP16; Lane 3: scFvR4-caspase3; Lane 4: scFvR4-caspase3(C163S); Lane 5: scFvF8-caspase3.

FIG. 3B shows a time course of luciferase activation in the presence of specific antigen. Luciferase activities are measured at 12, 24, 36, 48 and 60 hours after co-transfection with RSVluc, pEF-βgal expression plasmids and the three different scFv fusion protein expression plasmids as shown. Triangles: scFvR4-caspase3(C163S); squares: scFvF8-caspase3; circles: scFvR4-caspase3.

FIG. 3C shows a time course of luciferase activation in the absence of specific antigen. Luciferase activities are measured as in FIG. 3B but cells are transfected in the absence of the β-galactosidase expression construct. Triangles: scFvR4-caspase3(C163S); squares: scFvF8-caspase3; circles: scFvR4-caspase3.

FIG. 4. Apoptosis Assay with CHO Cells Expressing ScFv-Caspase and Specific Antigen

FIG. 4B shows results of experiments in which CHO cells are co-transfected with pEGFP-N1, expressing green fluorescent protein, and clones encoding various scFv fusions. Transfected cells are identified by fluorescence and sorted using a FACScalibur cell sorter. Genomic DNA is extracted from selected cells and PCR amplifications are carried out using ApoAlert LM-PCR Ladder Assay Kit. The products are fractionated on 1.5% agarose gels and visualised by staining with ethidium bromide. The lanes correspond to PCR reactions from DNA of cells which have been transfected with plasmids expressing scFvR4-VP16+β-gal (lane 1), scFvR4-caspase3+β gal (lane 2), scFvR4-caspase3(C163S)+βgal (lane 3), scFvF8-caspase3+βgal (lane 4) and scFvR4-caspase3 alone (lane 5). The negative PCR control represents a product from a reaction without template DNA.

FIG. 4B shows results of PCR reactions which are performed with the same DNA samples as in FIG. 4A, but using primers specific for Chinese hamster actin gene. The negative control as in FIG. 4A; the positive control is the reaction product obtained from a PCR amplification with purified CHO genomic DNA.

Size markers are a mixture of λ DNA digested with HindIII and _{ψ}X174 DNA digested with HaeIII.

FIG. 5. Effect of Anti-HIV Integrase-Caspase 3 Fusion on β-Galactosidase-Integrase Activity

FIG. 5A is a diagram depicting how apoptosis may be triggered by specific interaction between an anti-HIV integrase scFvIN33-caspase3 fusion and the HIV-1 integrase moieties fused to the β-galactosidase tetramer.

FIG. 5B shows results of an experiment in which CHO cells are transfected with plasmids expressing the various proteins as indicated in the presence of an expression clone encoding the HIV integrase epitope fused to β-galactosidase (HIVIN-βgal) or in the presence of a clone expressing wild type β-galactosidase (clone pEF-βgal). 60 hours after transfection, cell extracts are made and β-galactosidase levels are measured. Data is presented as relative β-galactosidase levels, with 100% being the level obtained when the respective β-galactosidase expressing clones (HIVIN-βgal and pEF-βgal) are co-transfected with pEF-BOS vector (empty vector) as 100%.

Front row HIVIN-βgal with 1. pEF-BOS; 2. ScFvIN33-caspase 3; 3. ScFvIN33-caspase 3(C163S); 4. ScFvR4-caspase 3; 5. ScFvF8-caspase 3. Back row is the same as the front row, except that pEF-βgal is co-transfected instead of HIVIN-βgal.

### DETAILED DESCRIPTION OF THE INVENTION

The methods of our invention allow the induction of a signal in a cell containing an entity, the detection of an entity within a cell, and the killing of a cell containing an entity. Our invention relies on the binding of first and second reporters to the entity. Thus, when the first reporter is brought into stable interaction with the second reporter through binding of the first and second reporters to the entity, a signal is generated. Advantageously, a stable interaction between the first and second reporters does not occur unless the reporters are brought together through binding of the reporters to their respective targets. The signal may be detected if desired. The first and second reporters are therefore two parts of a signal-generating agent, and which are capable of generating a signal by interacting.

It should be noted that our methods may be used generally to identify any cell, whether normal or abnormal (for example a cancerous or diseased cell). Generally, therefore, our methods may be used to detect any protein, nucleic acid or other entity present within a cell, or even to determine the sub-cellular localisation of any entity within a cell. Our methods are therefore capable of detecting the condition of a cell, according to the presence or absence of an entity associated with that condition. If it is desired that a cell which has been identified be eliminated, then this may be done by conventional means. Alternatively and preferably, the binding of the reporters may lead directly to the activation of a cell killing mechanism, so that the signal is the death of the cell. Advantageously, death of the cell results from apoptosis or programmed cell death.

Apoptosis is carried out by a family of cysteine proteases known as caspases that recognise specific amino acid sequences and cleave target proteins after an aspartic acid (Thornberry et al., 1998, Science 281, 1312-6). One member of the caspase family, caspase 3, is the so-called executioner in the apoptotic pathway and is responsible for the proteolytic cleavage of many proteins that are important in maintaining the integrity of living cells (Earnshaw et al., 1999, Ann. Rev. Biochem. 68, 383-424). Caspase-3 is synthesised as zymogen and is cleaved and activated by the initiator or upstream caspases such as caspase 8 (Srinivasula et al. (1996), Proc Natl Acad Sci U S A 93, 14486-91) and caspase 9 (Li et al., 1997, Cell 91, 479-89) to form an active tetrameric enzyme (Rotonda et al., 1996, Nat Struct Biol 3, 619-25). It has been shown that when two molecules of caspase 3 are brought into close proximity, by forced dimerisation, they can undergo self-activation and irreversibly lead to cell death (Colussi et al., 1998, J Biol Chem 273, 26566-70; MacCorkle et al, 1998, Proc Natl Acad Sci U S A 95, 3655-60; Fan et al., 1999, Hum Gene Ther 10, 2273-85).

Accordingly, in a preferred embodiment of the invention, the first reporter and the second reporter comprise caspase 3 molecules. Binding of the first and second reporters to the entity leads to stable interaction between the first and second reporters, self-activation of the caspase 3 molecules and generation of caspase activity. This leads to triggering of apoptosis within the cell. As described below, the first and second reporters advantageously bind to their respective targets via an antibody, and in our preferred embodiment, the caspase 3 is provided in the form of an intracellular antibody-caspase 3 fusion protein.

As used here, a “caspase molecule” or “caspase” includes a polypeptide sequence comprising some or all of the polypeptide sequence of a caspase, so long as caspase activity is retained. Thus, the first and second molecules may include polypeptide sequence from a caspase, together with other polypeptide sequence. The caspase sequence may form a moiety in or on the molecule.

The polypeptide sequence of the caspase may be altered with conservative amino acid substitutions which do not substantially affect the activity and function of the protein. Alternatively and preferably, mutations may be introduced which diminish the auto-toxicity of the caspase, and/or to make the caspase more effective in activating apoptosis. The caspase molecules may also be engineered so as to prevent or lower the tendency of the caspase molecules to homodimerise in the absence of the entity to be detected. Such variants and mutants of caspase may be made, and tested for effectiveness, by methods known in the art.

The signal maybe the emission or absorption of any electromagnetic (EM) radiation, for example, light. Included are fluorescence, phosphorescence or other signals which involve the modulation of the intensity or frequency of emission or absorption of radiation, for example, a FRET signal (described in further detail below). The first reporter and second reporter may each comprise a fluorophore such as a fluorescent protein or fluorescent chemical. Examples of fluorescent chemicals include allophycocyanine, phycocyanine, phycoerythrine, rhodamine, tetramethyl rhodamine, 7-nitro-benzofurazan rhodamine isothiocyanate, oxazine, coumarin, fluorescein derivatives, for example, FAM (6-carboxy-fluorescein), TET (6-carboxy-4,7,2′,7′-tetrachloro-fluorescein), (FITC) fluorescein isothiocyanate and carboxyfluorescein diacetate, as well as Texas Red, acridine yellow/orange, ethidium bromide, propidium iodide and bis-benzamide (commercially available from Hoechst under the trade name H33258).

Preferably, the first and second reporters comprise fluorescent polypeptides. Examples of fluorescent polypeptides and proteins include Green Fluorescent Protein (GFP) from Aequorea victoria and Red Fluorescent Protein (RFP) from Discosoma spp. Derivatives and variants of these proteins, such as Cyan Fluorescent Protein, Blue Fluorescent Protein, Enhanced Green Fluorescent Protein (EGFP; GFPmut1; Yang, T. T., et al. (1996) Nucleic Acids Res. 24(22):4592-4593;. Cormack, B. P., et al. (1996) Gene 173:33-38.), Enhanced Blue Fluorescent Protein (EBFP), Enhanced Yellow Fluorescent Protein (EYFP; Ormö, et al. (1996) Science 273:1392-1395), Destablised Enhanced Green Fluorescent Protein (d2EGFP; Living Colors Destabilized EGFP Vectors (April 1998) CLONTECHniques XIII(2):16-17), Enhanced Cyan Fluorescent Protein (ECFP), and GFPuv (Haas, J., et al. (1996) Curr. Biol. 6:315-324). may also be used. These fluorescent proteins are available from CLONTECH Laboratories, Inc. (Palo Alto, Calif., USA). Alternatively, the first and second reporters comprise polypeptide domains of a fluorescent polypeptide.

The signal may be a luminescence inducing activity. It will be appreciated that as light is generated during luminescence, the signal may at the same time be a luminescence inducing activity and emission of electromagnetic radiation.

The signal may also be the generation of an enzymatic activity, for example, transcriptional activity. The transcriptional activity may be detected by assaying the expression of a reporter gene such as CD4, by fluorescent antibodies and FACs for example.

Alternatively, the signal may be detected as cell growth, cell division or differentiation. The first and second reporters may stably interact to produce a transcriptional activity, which may activate a program of differentiation or cell growth. For example, VEGF (vascular endothelial growth factor) and/or other angiogenic growth factors may also be expressed to induce angiogenesis. It has been shown that the Lmo2 LIM-only protein is specifically needed for angiogenesis (Yamada et al., 2000, Proc Natl Acad Sci USA 97, 320-324), and accordingly the transcriptional activity may be Lmo2 activity. Cell growth and detection may be detected by microscopy, detecting appropriate cell surface markers, etc as known in the art.

The cell may be any prokaryotic or eukaryotic cell. Preferably, the cell is an animal cell or a human cell; most preferably, the cell is a human cell. The entity may be present in any cellular compartment, preferably the cytoplasm or nucleus.

The cell may be a normal cell, and our methods may therefore be used as a means of tissue typing, for example, by detecting an entity associated with a particular developmental lineage. Alternatively, the methods may be used as a means of determining the condition of a cell, for example, whether a cell is cancerous, by detecting a tumour associated entity within the cell. Thus, the entity may be a mutant oncogenic protein or polypeptide, derived from a wild type protein by a point mutation, deletion, insertion or a chromosomal translocation in a nucleic acid encoding the polypeptide. An example is p21 ras, which results from one or more mutations in a corresponding wild type protein. Mutations in tumour suppressor genes are often associated with cancer cells, and the proteins they give rise to, for example, mutant p53 protein, may also be detected using the methods of our invention. Alternatively, the oncogenic protein may be a chimaeric fusion protein resulting from a chromosomal translocation, such as the BCR-ABL fusion arising from a Philadelphia translocation.

The entity may also be a protein or nucleic acid associated with an abnormal cell. Such a cell may express aberrant proteins not found in a normal cell, for example, viral or bacterial specific proteins expressed as a result of infection. For example, detection of the Human Immunodeficiency Virus (HIV) integrase protein will allow determination of whether a cell is infected with HIV. Thus, our methods may be used to determine whether a cell is diseased, infected or abnormal. Alternatively, and preferably, the diseased, infected or abnormal cell is killed by activation of apoptosis as described above. It will be appreciated that in this case, the signal does not need to be detected at all.

It will be appreciated that it is also possible to detect a protein by detecting a corresponding nucleic acid giving rise to the protein. For example, a messenger RNA or a DNA sequence carrying a mutation and encoding a tumour associated protein may be detected.

In a preferred embodiment, the first and second reporters comprise binding means, which binding means are immunoglobulins provided by expressing nucleic acids within the cell. The nucleic acids may be localised to a subcellular compartment suitable for detecting the entity. For example, if the entity is a cytoplasmic protein, the nucleic acids are localised to the cytoplasm of the cell, and transcribed and/or translated there. The molecules may also be localised to any desired subcellular compartment, such as the nucleus (for example by fusion to a nuclear localisation signal), to the ER, using an ER retention signal, to the mitochondria using a mitochondria-targeting sequence (MTS), the plasma membrane or other locations such as the plasma membrane. Such targeting sequences are reviewed generally in Baker et al., 1996, Biol Rev Camb Philos Soc 71, 637-702.

Mitochondrial targeting sequences and mechanisms for directing and translocating proteins through the mitochondrial membranes, are discussed in, for example, Omura, 1998, Biochem (Tokyo) 123, 1010-6, Glaser et al., 1998, Plant Mol Biol, 38, 311-38 and Voos et al., 1999, Biochim Biophys Acta 1422, 235-54. Mitochondrial targeting of bioactive compounds is reviewed in Murphy, 1997, Trends Biotechnol 15, 326-30. Since mitochondria are involved in many critical cell processes, our invention may be used to detect an entity comprising an abnormal mitochondrial protein or DNA associated with mitochondrial DNA diseases. Cells containing aberrant mitochondrial protein or mutant mtDNA may be detected, and optionally killed.

Nuclear localisation sequences include the SV40 large T antigen consensus sequence PKKKRKV (reviewed in Dingwall, et al., 1991, Trends Biochem. Sci. 16, 478-481), or the bipartite nuclear localisation sequence as exemplified by nucleoplasmin protein (Dingwall, et al., 1987, EMBO J. 6, 69-74; Robbins, et al 1991, Cell 64,615-623).

The reporter(s) may also be targeted to the plasma membrane, in order to detect an entity which is present in the plasma membrane, such as a membrane protein. It is known that the p21-ras oncogene gene product is localised at the plasma membrane, and such targeted reporter(s) may suitably be used to detect the ras protein. Targeting to the plasma membrane may be achieved by linking the reporter(s) to a transmembrane protein, or a portion of the transmembrane protein responsible for membrane localisation, such as a transmembrane α helix as known in the art.

Where the reporters are expressed as recombinant proteins, the nuclear targeting sequences, mitochondrial targeting sequences, ER retention signals etc may be engineered into the reporter by cloning the suitable sequence into the expression construct.

Nucleic acids encoding immunoglobulins may be obtained from libraries encoding a multiplicity of such molecules. For example, phage display libraries of antibody molecules are known and may be used in this process. Advantageously, the library encodes a repertoire of immunoglobulin molecules. Methods for generating repertoires are well characterised in the art.3

Libraries may moreover be constructed from nucleic acids isolated from organisms which have been challenged with an antigen. Antigen challenge will normally result in the generation of a polyclonal population of immunoglobulins, each of which is capable of binding to the antigen but which may differ from the others in terms of epitope specificity or other features. By cloning antibody genes from an organism a polyclonal population of immunoglobulins may be subjected to selection in order to isolate immunoglobulins which are suitable for use in our methods.

As noted above, the first and/or second reporters may comprise association means, which may comprise a polypeptide. Where the association means is a polypeptide, one or both of the first and second reporters may be provided in the form of a fusion protein comprising the immunoglobulin and the polypeptide. In a preferred embodiment of the invention, the fusion protein is an intracellular antibody-caspase 3 fusion. One or both fusion proteins may be expressed from appropriate nucleic acid constructs, which are transcribed to produce first or second immunoglobulin together with first or second polypeptides. The nucleic acid construct(s) may be targeted intracellularly as described above. The nucleic acid constructs may be expression vectors capable of directing expression of the nucleic acid encoding the fusion protein in the cell in which the methods of the invention is to be performed.

Binding Moieties

The reporters according to the invention preferably comprise moieties which are capable of binding to cellular entities as described. These may be immunoglobulins, particularly intracellular antibodies as described below; however, the invention also includes the use of polypeptides and nucleic acid binding molecules which are capable of binding targets within a cell. Such binding molecules have the advantage of typically being smaller than antibodies, and thus better able to target a reporter to entities within a cell.

The invention thus provides the use of target-specific binding polypeptides and/or nucleic acid aptamers to direct the first and/or second reporter to one or more cellular targets. As used herein, a “target-specific binding polypeptide and/or nucleic acid aptamer” is a polypeptide or nucleic acid molecule which is capable of specific binding to a molecular target in a cell. Such peptides or aptamers may be used in place of immunoglobulins to achieve targeting or reporters to entities in cells in accordance with the invention.

Polypeptides having binding activity may be developed, for example, from recombinant libraries of random polypeptide structures. Selection of polypeptides having binding affinity for a desired target by techniques such as phage display, SELEX, mRNA display or surface plasmon resonance, followed if necessary by refinement of the binding specificity and affinity by repeated rounds of mutation and selection, are techniques known to those skilled in the art.

For example, selection of binding polypeptides by mRNA selection is described by Wilson et al., Proc Natl Acad Sci USA Mar. 27, 2001;98(7):3750-3755. Srebalus and Clemmer, Proc Natl Acad Sci USA Mar. 27, 2001;98(7):3750-3755, describe the use of MALDI-TOFMS to characterise the binding of a library of polypeptides to a target molecule. The use of phage display is reviewed by Nilsson et al., Adv Drug Deliv Rev Sep. 30, 2000;43(2-3):165-96, and McGregor, Mol Biotechnol October 1996;6(2):155-62. The use of nucleic acid aptamers is reviewed by Hermann and Patel, Science Feb. 4, 2000;287(5454):820-5. SELEX is a method for the in vitro evolution of nucleic acid molecules with highly specific binding to target molecules. It is described, for example, in U.S. Pat. Nos. 5,654,151, 5,503,978, 5,567,588 and 5,270,163, as well as PCT publication WO 96/38579.

Iterative selection procedures such as phage display and SESLX are based on the principle that within a library containing a large number of possible sequences and structures there is a wide range of binding affinities for a given target. A library comprising, for example a 20 subunit randomised polypeptide or nucleic acid polymer can have 4²⁰ structural possibilities. Those which have the higher affinity constants for the target are considered to be most likely to bind. The process of partitioning, dissociation and amplification generates a second nucleic acid library, enriched for the higher binding affinity candidates. Additional rounds of selection progressively favour the best ligands until the resulting library is predominantly composed of only one or a few sequences. These can then be cloned, sequenced and individually tested for binding affinity as pure ligands.

Cycles of selection and mutation/amplification are repeated until a desired goal is achieved. In the most general case, selection/amplification is continued until no significant improvement in binding strength is achieved on repetition of the cycle. The iterative selection/amplification method is sensitive enough to allow isolation of a single sequence variant in a library containing at least 10¹⁴ sequences. The method could, in principle, be used to sample as many as about 10¹⁸ different nucleic acid species. The members of the library preferably include a randomised sequence portion as well as conserved sequences necessary for efficient amplification. Sequence variants can be produced in a number of ways including synthesis of randomised nucleic acid sequences and size selection from randomly cleaved cellular nucleic acids. The variable sequence portion may contain fully or partially random sequence; it may also contain subportions of conserved sequence incorporated with randomised sequence. Sequence variation in test nucleic acids can be introduced or increased by mutagenesis before or during the selection/amplification iterations and by specific modification.

Methods for the selection of binding polypeptides and aptamers are further described below. In general techniques for the selection of immunoglobulin molecules may readily be adapted to the selection of peptides for use in the present invention.

Immunoglobulins

Immunoglobulin molecules are in the broadest sense members of the immunoglobulin superfamily, a family of polypeptides which comprise the immunoglobulin fold characteristic of antibody molecules, which contains two P sheets and, usually, a conserved disulphide bond. Members of the immunoglobulin superfamily are involved in many aspects of cellular and non-cellular interactions in vivo, including widespread roles in the immune system (for example, antibodies, T-cell receptor molecules and the like), involvement in cell adhesion (for example the ICAM molecules) and intracellular signalling (for example, receptor molecules, such as the PDGF receptor). The methods of the present invention may therefore make use of any immunoglobulin superfamily molecule which is capable of binding to a target. Peptides or fragments derived from immunoglobulins may also be used.

Antibodies, as used herein, refers to complete antibodies or antibody fragments capable of binding to a selected target, and including Fv, ScFv, Fab′ and F(ab′)₂, monoclonal and polyclonal antibodies, engineered antibodies including chimeric, CDR-grafted and humanised antibodies, and artificially selected antibodies produced using phage display or alternative techniques. Small fragments, such as Fv and ScFv, possess advantageous properties for diagnostic and therapeutic applications on account of their small size and consequent superior tissue distribution. Preferably, the antibody is a single chain antibody or scFv.

The antibodies may be altered antibodies comprising an effector protein such as a toxin or a label. Use of labelled antibodies allows the imaging of the distribution of the antibody in vivo. Such labels may be radioactive labels or radioopaque labels, such as metal particles, which are readily visualisable within the body of a patient. Moreover, they may be fluorescent labels (such as the ones described here) or other labels which are visualisable on tissue samples removed from patients. Antibodies with effector groups may be linked to any association means as described above.

Antibodies may be obtained from animal serum, or, in the case of monoclonal antibodies or fragments thereof, produced in cell culture. Recombinant DNA technology may be used to produce the antibodies according to established procedure, in bacterial, yeast, insect or preferably mammalian cell culture. The selected cell culture system preferably secretes the antibody product.

Multiplication of hybridoma cells or mammalian host cells in vitro is carried out in suitable culture media, which are the customary standard culture media, for example Dulbecco's Modified Eagle Medium (DMEM) or RPMI 1640 medium, optionally replenished by a mammalian serum, e.g. foetal calf serum, or trace elements and growth sustaining supplements, e.g. feeder cells such as normal mouse peritoneal exudate cells, spleen cells, bone marrow macrophages, 2-aminoethanol, insulin, transferrin, low density lipoprotein, oleic acid, or the like. Multiplication of host cells which are bacterial cells or yeast cells is likewise carried out in suitable culture media known in the art, for example for bacteria in medium LB, NZCYM, NZYM, NZM, Terrific Broth, SOB, SOC, 2×YT, or M9 Minimal Medium, and for yeast in medium YPD, YEPD, Minimal Medium, or Complete Minimal Dropout Medium.

Use of insect cells as hosts for the expression of proteins has advantages in that the cloning and expression process is relatively easy and quick. In addition, there is a high probability of obtaining a correctly folded and biologically active protein when compared to bacterial or yeast expression. Insect cells may be cultured in serum free medium, which is cheaper and safer compared to serum containing medium. Recombinant baculovirus may be used as an expression vector, and the construct used to transfect a host cell line, which may be any of a number of lepidopteran cell lines, in particular Spodoptera frugiperda Sf9, as known in the art. Reviews of expression of recombinant proteins in insect host cells are provided by Altmann et al. (1999), Glycoconj J 1999, 16, 109-23 and Kost and Condreay (1999), Curr Opin Biotechnol, 10, 428-33.

In vitro production provides relatively pure antibody preparations and allows scale-up to give large amounts of the desired antibodies. Techniques for bacterial cell, yeast, insect and mammalian cell cultivation are known in the art and include homogeneous suspension culture, e.g. in an airlift reactor or in a continuous stirrer reactor, or immobilised or entrapped cell culture, e.g. in hollow fibres, microcapsules, on agarose microbeads or ceramic cartridges.

Large quantities of the desired antibodies can also be obtained by multiplying mammalian cells in vivo. For this purpose, hybridoma cells producing the desired antibodies are injected into histocompatible mammals to cause growth of antibody-producing tumours. Optionally, the animals are primed with a hydrocarbon, especially mineral oils such as pristane (tetramethyl-pentadecane), prior to the injection. After one to three weeks, the antibodies are isolated from the body fluids of those mammals. For example, hybridoma cells obtained by fusion of suitable myeloma cells with antibody-producing spleen cells from Balb/c mice, or transfected cells derived from hybridoma cell line Sp2/0 that produce the desired antibodies are injected intraperitoneally into Balb/c mice optionally pre-treated with pristane, and, after one to two weeks, ascitic fluid is taken from the animals.

The foregoing, and other, techniques are discussed in, for example, Kohler and Milstein, (1975) Nature 256:495-497; U.S. Pat. No. 4,376,110; Harlow and Lane, Antibodies: a Laboratory Manual, (1988) Cold Spring Harbor, incorporated herein by reference. Techniques for the preparation of recombinant antibody molecules is described in the above references and also in, for example, EP 0623679; EP 0368684 and EP 0436597, which are incorporated herein by reference.

The cell culture supernatants are screened for the desired antibodies, preferentially by immunofluorescent staining of cells expressing the desired target by immunoblotting, by an enzyme immunoassay, e.g. a sandwich assay or a dot-assay, or a radioimmunoassay.

For isolation of the antibodies, the immunoglobulins in the culture supernatants or in the ascitic fluid may be concentrated, e.g. by precipitation with ammonium sulphate, dialysis against hygroscopic material such as polyethylene glycol, filtration through selective membranes, or the like. If necessary and/or desired, the antibodies are purified by the customary chromatography methods, for example gel filtration, ion-exchange chromatography, chromatography over DEAE-cellulose and/or immunoaffinity chromatography, e.g. affinity chromatography with the a protein containing a target or with Protein-A.

Antibodies generated according to the foregoing procedures may be cloned by isolation of nucleic acid from cells, according to standard procedures. Usefully, nucleic acids variable domains of the antibodies may be isolated and used to construct antibody fragments, such as scFv.

The invention therefore preferably employs recombinant nucleic acids comprising an insert coding for a heavy chain variable domain and/or for a light chain variable domain of antibodies. By definition such nucleic acids comprise coding single stranded nucleic acids, double stranded nucleic acids consisting of said coding nucleic acids and of complementary nucleic acids thereto, or these complementary (single stranded) nucleic acids themselves.

Furthermore, nucleic acids encoding a heavy chain variable domain and/or for a light chain variable domain of antibodies can be enzymatically or chemically synthesised nucleic acids having the authentic sequence coding for a naturally-occurring heavy chain variable domain and/or for the light chain variable domain, or a mutant thereof. A mutant of the authentic sequence is a nucleic acid encoding a heavy chain variable domain and/or a light chain variable domain of the above-mentioned antibodies in which one or more amino acids are deleted or exchanged with one or more other amino acids. Preferably said modification(s) are outside the CDRs of the heavy chain variable domain and/or of the light chain variable domain of the antibody. Such a mutant nucleic acid is also intended to be a silent mutant wherein one or more nucleotides are replaced by other nucleotides with the new codons coding for the same amino acid(s). Such a mutant sequence is also a degenerated sequence. Degenerated sequences are degenerated within the meaning of the genetic code in that an unlimited number of nucleotides are replaced by other nucleotides without resulting in a change of the amino acid sequence originally encoded. Such degenerated sequences may be useful due to their different restriction sites and/or frequency of particular codons which are preferred by the specific host, particularly yeast, bacterial or mammalian cells, to obtain an optimal expression of the heavy chain variable domain and/or a light chain variable domain.

The term mutant is intended to include a DNA mutant obtained by in vitro or in vivo mutagenesis of DNA according to methods known in the art.

Recombinant DNA technology may be used to improve the antibodies of the invention. Thus, chimeric antibodies may be constructed in order to decrease the immunogenicity thereof in diagnostic or therapeutic applications. Moreover, immunogenicity may be minimised by humanising the antibodies by CDR grafting [European Patent 0 239 400 (Winter)] and, optionally, framework modification [European Patent 0239400; Riechmann et al., (1988) Nature 322:323-327; and as reviewed in international patent application WO 90/07861 (Protein Design Labs)].

The invention therefore also employs recombinant nucleic acids comprising an insert coding for a heavy chain variable domain of an antibody fused to a human constant domain γ, for example γ1, γ2, γ3 or γ4, preferably γ1 or γ4. Likewise the invention concerns recombinant DNAs comprising an insert coding for a light chain variable domain of an antibody fused to a human constant domain κ or λ, preferably κ.

More preferably, the invention employs CDR-grafted antibodies, which are preferably CDR-grafted light chain and heavy chain variable domains only. Advantageously, the heavy chain variable domain and the light chain variable domain are linked by way of a spacer group, optionally comprising a signal sequence facilitating the processing of the antibody in the host cell and/or a DNA coding for a peptide facilitating the purification of the antibody and/or a cleavage site and/or a peptide spacer and/or an effector molecule. Such antibodies are known as scFvs.

Antibodies may moreover be generated by mutagenesis of antibody genes to produce artificial repertoires of antibodies. This technique allows the preparation of antibody libraries, as discussed further below; antibody libraries are also available commercially. Hence, the present invention advantageously employs artificial repertoires of immunoglobulins, preferably artificial ScFv repertoires, as an immunoglobulin source.

Isolated or cloned antibodies may be linked to other molecules, for example nucleic acid or protein association means by chemical coupling, using protocols known in the art (for example, Harlow and Lane, Antibodies: a Laboratory Manual, (1988) Cold Spring Harbor, and Maniatis, T., Fritsch, E. F. and Sambrook, J. (1991), Molecular Cloning: A Laboratory Manual. Cold Spring Harbor, N.Y., Cold Spring Harbor Laboratory Press).

Libraries and Selection Systems

Immunoglobulins for use in the invention may be isolated from libraries comprising artificial repertoires of immunoglobulin polypeptides. Advantageously, the immunoglobulins may be pre-selected by screening against the desired target, such that the methods of the invention are performed with immunoglobulins which substantially all are specific for the intended target.

Any library selection system may be used in conjunction with the invention. Selection protocols for isolating desired members of large libraries are known in the art, as typified by phage display techniques. Such systems, in which diverse peptide sequences are displayed on the surface of filamentous bacteriophage (Scott and Smith (1990 supra), have proven useful for creating libraries of antibody fragments (and the nucleotide sequences that encoding them) for the in vitro selection and amplification of specific antibody fragments that bind a target antigen. The nucleotide sequences encoding the V_{H} and V_{L} regions are linked to gene fragments which encode leader signals that direct them to the periplasmic space of E. coli and as a result the resultant antibody fragments are displayed on the surface of the bacteriophage, typically as fusions to bacteriophage coat proteins (e.g., pIII or pVIII). Alternatively, antibody fragments are displayed externally on lambda phage capsids (phagebodies). An advantage of phage-based display systems is that, because they are biological systems, selected library members can be amplified simply by growing the phage containing the selected library member in bacterial cells. Furthermore, since the nucleotide sequence that encodes the polypeptide library member is contained on a phage or phagemid vector, sequencing, expression and subsequent genetic manipulation is relatively straightforward.

Methods for the construction of bacteriophage antibody display libraries and lambda phage expression libraries are well known in the art (McCafferty et al. (1990) supra; Kang et al. (1991) Proc. Natl. Acad. Sci. U.S.A., 88: 4363; Clackson et al. (1991) Nature, 352: 624; Lowman et al. (1991) Biochemistry, 30: 10832; Burton et al. (1991) Proc. Natl. Acad. Sci U.S.A., 88: 10134; Hoogenboom et al. (1991) Nucleic Acids Res., 19: 4133; Chang et al. (1991) J. Immunol., 147: 3610; Breitling et al. (1991) Gene, 104: 147; Marks et al. (1991) supra; Barbas et al. (1992) supra; Hawkins and Winter (1992) J. Immunol., 22: 867; Marks et al., 1992, J. Biol. Chem., 267: 16007; Lerner et al. (1992) Science, 258: 1313, incorporated herein by reference).

One particularly advantageous approach has been the use of scFv phage-libraries (Bird, R. E., et al. (1988) Science 242: 423-6, Huston et al., 1988, Proc. Natl. Acad. Sci U.S.A., 85: 5879-5883; Chaudhary et al. (1990) Proc. Natl. Acad. Sci U.S.A., 87: 1066-1070; McCafferty et al. (1990) supra; Clackson et al. (1991) supra; Marks et al. (1991) supra; Chiswell et al. (1992) Trends Biotech., 10: 80; Marks et al. (1992) supra). Various embodiments of scFv libraries displayed on bacteriophage coat proteins have been described. Refinements of phage display approaches are also known, for example as described in WO96/06213 and WO92/01047 (Medical Research Council et al.) and WO97/08320 (Morphosys, supra), which are incorporated herein by reference.

Alternative library selection technologies include bacteriophage lambda expression systems, which may be screened directly as bacteriophage plaques or as colonies of lysogens, both as previously described (Huse et al. (1989) Science, 246: 1275; Caton and Koprowski (1990) Proc. Natl. Acad. Sci. U.S.A., 87; Mullinax et al. (1990) Proc. Natl. Acad. Sci. U.S.A., 87: 8095; Persson et al. (1991) Proc. Natl. Acad. Sci. U.S.A., 88: 2432) and are of use in the invention. These expression systems may be used to screen a large number of different members of a library, in the order of about 10⁶ or even more. Other screening systems rely, for example, on direct chemical synthesis of library members. One early method involves the synthesis of peptides on a set of pins or rods, such as described in WO84/03564. A similar method involving peptide synthesis on beads, which forms a peptide library in which each bead is an individual library member, is described in U.S. Pat. No. 4,631,211 and a related method is described in WO92/00091. A significant improvement of the bead-based methods involves tagging each bead with a unique identifier tag, such as an oligonucleotide, so as to facilitate identification of the amino acid sequence of each library member. These improved bead-based methods are described in WO93/06121.

Another chemical synthesis method involves the synthesis of arrays of peptides (or peptidomimetics) on a surface in a manner that places each distinct library member (e.g., unique peptide sequence) at a discrete, predefined location in the array. The identity of each library member is determined by its spatial location in the array. The locations in the array where binding interactions between a predetermined molecule (e.g., a receptor) and reactive library members occur is determined, thereby identifying the sequences of the reactive library members on the basis of spatial location. These methods are described in U.S. Pat. No. 5,143,854; WO90/15070 and WO92/10092; Fodor et al. (1991) Science, 251: 767; Dower and Fodor (1991) Ann. Rep. Med. Chem., 26: 271.

Other systems for generating libraries of polypeptides or nucleotides involve the use of cell-free enzymatic machinery for the in vitro synthesis of the library members. In one method, RNA molecules are selected by alternate rounds of selection against a target ligand and PCR amplification (Tuerk and Gold (1990) Science, 249: 505; Ellington and Szostak (1990) Nature, 346: 818). A similar technique may be used to identify DNA sequences which bind a predetermined human transcription factor (Thiesen and Bach (1990) Nucleic Acids Res., 18: 3203; Beaudry and Joyce (1992) Science, 257: 635; WO92/05258 and WO92/14843). In a similar way, in vitro translation can be used to synthesise polypeptides as a method for generating large libraries. These methods which generally comprise stabilised polysome complexes, are described further in WO88/08453, WO90/05785, WO90/07003, WO91/02076, WO91/05058, and WO92/02536. Alternative display systems which are not phage-based, such as those disclosed in WO95/22625 and WO95/11922 (Affymax) use the polysomes to display polypeptides for selection. These and all the foregoing documents also are incorporated herein by reference.

An alternative to the use of phage or other cloned libraries is to use nucleic acid, preferably RNA, derived from the spleen of an animal which has been immunised with the selected target. RNA thus obtained represents a natural library of immunoglobulins. Isolation of V-region and C-region mRNA permits antibody fragments, such as Fab or Fv, to be expressed intracellularly in accordance with the invention. Briefly, RNA is isolated from the spleen of an immunised animal and PCR primers used to amplify V_{H} and V_{L} cDNA selectively from the RNA pool. The V_{H} and V_{L} sequences thus obtained are joined to make scFv antibodies. PCR primer sequences are based on published V_{H} and V_{L} sequences and are available commercially in kit form.

A preferred aspect of the present invention is the use of intracellular immunoglobulins, for example intracellular antibodies. Intracellular antibodies or intrabodies have been demonstrated to function in antigen recognition in the cells of higher organisms (reviewed in Cattaneo, A. & Biocca, S. (1997) Intracellular Antibodies: Development and Applications. Landes and Springer-Verlag). This interaction can influence the function of cellular proteins which have been successfully inhibited in the cytoplasm, the nucleus or in the secretory pathway. This efficacy has been demonstrated for viral resistance in plant biotechnology (Tavladoraki, P., et al. (1993) Nature 366: 469-472) and several applications have been reported of intracellular antibodies binding to HIV viral proteins (Mhashilkar, A. M., et al. (1995) EMBO J. 14: 1542-51; Duan, L. & Pomerantz, R. J. (1994) Nucleic Acids Res 22: 5433-8; Maciejewski, J. P., et al. (1995) Nat Med 1: 667-73; Levy-Mintz, P., et al. (1996) J. Virol. 70: 8821-8832) and to oncogene products (Biocca, S., Pierandrei-Amaldi, P. & Cattaneo, A. (1993) Biochem Biophys Res Commun 197: 422-7; Biocca, S., Pierandrei-Amaldi, P., Campioni, N. & Cattanco, A. (1994) Biotechnology (N Y) 12: 396-9; Cochet, O., et al. (1998) Cancer Res 58: 1170-6).

A method for the selection of intracellular immunoglobulins from scFv libraries has been described (see International patent application WO 0054057). This selection method (see also Visintin, M., Tse, E., Axelson, H., Rabbitts, T. H. and Cattaneo, A. (1999). Selection of antibodies for intracellular function using a two-hybrid in vivo system. Proc. Natl. Acad. Sci. USA 96, 11723-11728) takes advantage of the ability of protein interactions to be detected in cellular environments, such as shown for the yeast two-hybrid system. It is based on the fact that some antibody scFv fragments can fold adequately in vivo to bind antigen in a VH-VL dependent way (i.e. via the antibody combining site) and thus using a library of diverse antibody specificities facilitates their identification, if sufficient scFv are screened. The screening system comprises the yeast cell expression of a “bait” antigen fused to the LexA DNA binding domain and a library of “prey” scFv fused to the VP16 transcription activation domain. Interaction between the antigen bait and any specific antibody scFv fragment in the yeast intracellular environment results in the formation of a protein complex in which the DNA binding domain and the activation domain are in close proximity. This results in the activation of yeast chromosomal reporter genes such as HIS3 and LacZ, facilitating the identification and thus isolation of the yeast carrying the DNA vectors encoding the scFv, which in turn can be isolated to yield the DNA sequence of the antigen-specific scFv. The main limitation of this approach is the number of scFv-VP16 fusion preys that can be screened in yeast antibody-antigen interaction system (conveniently up to 2-5×10⁶). The effective library size may be increased, however, for example via the use of one or more round of in vitro phage scFv library screening (panning). For instance, using bacterially produced antigen which is coated on a surface, prior to the in vivo yeast antibody-antigen interaction screening.

Delivery of Reporters to Cells

In order for the first reporter and second reporter to bind to the entity within the cell, it is necessary to provide the reporters within the intracellular environment of the cell. Where the first and/or second reporter is a polypeptide (a “polypeptide reporter”), for example an antibody caspase 3 fusion protein, this is preferably achieved by transfecting the cell with appropriate nucleic acids which encode the first and/or second reporters. If either or both of the reporters consists of a nucleic acid (a “nucleic acid reporter”), then the nucleic acid itself may be transfected into the cell.

Nucleic acids encoding a polypeptide reporter can be incorporated into vectors for expression. As used herein, vector (or plasmid) refers to discrete elements that are used to introduce heterologous DNA into cells for expression thereof. Selection and use of such vehicles are well within the skill of the artisan. Many vectors are available, and selection of appropriate vector will depend on the intended use of the vector, the size of the nucleic acid to be inserted into the vector, and the host cell to be transformed with the vector. Each vector contains various components depending on its function and the host cell for which it is compatible. The vector components generally include, but are not limited to, one or more of the following: an origin of replication, one or more marker genes, an enhancer element, a promoter, a transcription termination sequence and a signal sequence.

The vector may also include one or more Internal Ribosome Entry Sites (IRES), and these site(s) are preferably arranged in the vector adjacent to one or more multiple cloning sequences (MCS) into each of which a gene may be inserted. Use of such a vector therefore allows more than one insert to be cloned into the expression construct, with transcription of the construct resulting in a bi- or poly-cistronic messenger RNA. Translation occurs from each of the ribosome binding sites, including the Internal Ribosome Entry Sites. This allows for more than one polypeptide to be expressed from a single expression construct.

Moreover, nucleic acids encoding a polypeptide reporter or a nucleic acid reporter, or any components of these, may be incorporated into cloning vectors, for general manipulation and nucleic acid amplification purposes.

Both expression and cloning vectors generally contain nucleic acid sequence that enable the vector to replicate in one or more selected host cells. Typically in cloning vectors, this sequence is one that enables the vector to replicate independently of the host chromosomal DNA, and includes origins of replication or autonomously replicating sequences. Such sequences are well known for a variety of bacteria, yeast and viruses. The origin of replication from the plasmid pBR322 is suitable for most Gram-negative bacteria, the 2 micron plasmid origin is suitable for yeast, and various viral origins (e.g. SV 40, polyoma, adenovirus) are useful for cloning vectors in mammalian cells. Generally, the origin of replication component is not needed for mammalian expression vectors unless these are used in mammalian cells competent for high level DNA replication, such as COS cells.

Most expression vectors are shuttle vectors, i.e. they are capable of replication in at least one class of organisms but can be transfected into another class of organisms for expression. For example, a vector is cloned in E. coli and then the same vector is transfected into yeast or mammalian cells even though it is not capable of replicating independently of the host cell chromosome. DNA may also be replicated by insertion into the host genome. However, the recovery of genomic DNA is more complex than that of exogenously replicated vector because restriction enzyme digestion is required to excise the nucleic acid. DNA can be amplified by PCR and be directly transfected into the host cells without any replication component.

Advantageously, an expression and cloning vector may contain a selection gene also referred to as selectable marker. This gene encodes a protein necessary for the survival or growth of transformed host cells grown in a selective culture medium. Host cells not transformed with the vector containing the selection gene will not survive in the culture medium. Typical selection genes encode proteins that confer resistance to antibiotics and other toxins, e.g. ampicillin, neomycin, methotrexate or tetracycline, complement auxotrophic deficiencies, or supply critical nutrients not available from complex media.

As to a selective gene marker appropriate for yeast, any marker gene can be used which facilitates the selection for transformants due to the phenotypic expression of the marker gene. Suitable markers for yeast are, for example, those conferring resistance to antibiotics G418, hygromycin or bleomycin, or provide for prototrophy in an auxotrophic yeast mutant, for example the URA3, LEU2, LYS2, TRP1, or HIS3 gene.

Since the replication of vectors is conveniently done in E. coli, an E. coli genetic marker and an E. coli origin of replication are advantageously included. These can be obtained from E. coli plasmids, such as pBR322, Bluescript® vector or a pUC plasmid, e.g. pUC 18 or pUC19, which contain both an E. coli replication origin and an E. coli genetic marker conferring resistance to antibiotics, such as ampicillin.

Suitable selectable markers for mammalian cells are those that enable the identification of cells expressing the desired nucleic acid, such as dihydrofolate reductase (DHFR, methotrexate resistance), thymidine kinase, or genes conferring resistance to G418 or hygromycin. The mammalian cell transformants are placed under selection pressure which only those transformants which have taken up and are expressing the marker are uniquely adapted to survive. In the case of a DHFR or glutamine synthase (GS) marker, selection pressure can be imposed by culturing the transformants under conditions in which the pressure is progressively increased, thereby leading to amplification (at its chromosomal integration site) of both the selection gene and the linked nucleic acid. Amplification is the process by which genes in greater demand for the production of a protein critical for growth, together with closely associated genes which may encode a desired protein, are reiterated in tandem within the chromosomes of recombinant cells. Increased quantities of desired protein are usually synthesised from thus amplified DNA.

Expression and cloning vectors usually contain a promoter that is recognised by the host organism and is operably linked to the desired nucleic acid. Such a promoter may be inducible or constitutive. The promoters are operably linked to the nucleic acid by removing the promoter from the source DNA and inserting the isolated promoter sequence into the vector. Both the native promoter sequence and many heterologous promoters may be used to direct amplification and/or expression of nucleic acid encoding the immunoglobulin, optionally together with an appropriate association means. The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences.

Promoters suitable for use with prokaryotic hosts include, for example, the β-lactamase and lactose promoter systems, alkaline phosphatase, the tryptophan (trp) promoter system and hybrid promoters such as the tac promoter. Their nucleotide sequences have been published, thereby enabling the skilled worker operably to ligate them a desired nucleic acid, using linkers or adaptors to supply any required restriction sites. Promoters for use in bacterial systems will also generally contain a Shine-Delgarno sequence operably linked to the nucleic acid.

Preferred expression vectors are bacterial expression vectors which comprise a promoter of a bacteriophage such as phage λ or T7 which is capable of functioning in the bacteria. In one of the most widely used expression systems, the nucleic acid encoding the fusion protein may be transcribed from the vector by T7 RNA polymerase (Studier et al, Methods in Enzymol. 185; 60-89, 1990). In the E. coli BL21(DE3) host strain, used in conjunction with pET vectors, the T7 RNA polymerase is produced from the λ-lysogen DE3 in the host bacterium, and its expression is under the control of the IPTG inducible lac UV5 promoter. This system has been employed successfully for over-production of many proteins. Alternatively the polymerase gene may be introduced on a lambda phage by infection with an int-phage such as the CE6 phage which is commercially available (Novagen, Madison, USA). other vectors include vectors containing the lambda PL promoter such as PLEX (Invitrogen, NL), vectors containing the trc promoters such as pTrcH is XpressTm (Invitrogen) or pTrc99 (Pharmacia Biotech, SE), or vectors containing the tac promoter such as pKK223-3 (Pharmacia Biotech) or PMAL (New England Biolabs, MA, USA).

Suitable promoter sequences for use with yeast hosts may be regulated or constitutive and are preferably derived from a highly expressed yeast gene, especially a Saccharomyces cerevisiae gene. Thus, the promoter of the TRP 1 gene, the ADHI or ADHII gene, the acid phosphatase (PH05) gene, a promoter of the yeast mating pheromone genes coding for the a- or α-factor or a promoter derived from a gene encoding a glycolytic enzyme such as the promoter of the enolase, glyceraldehyde-3-phosphate dehydrogenase (GAP), 3-phospho glycerate kinase (PGK), hexokinase, pyruvate decarboxylase, phosphofructokinase, glucose-6-phosphate isomerase, 3-phosphoglycerate mutase, pyruvate kinase, triose phosphate isomerase, phosphoglucose isomerase or glucokinase genes, the S. cerevisiae GAL 4 gene, the S. pombe nmt 1 gene or a promoter from the TATA binding protein (TBP) gene can be used. Furthermore, it is possible to use hybrid promoters comprising upstream activation sequences (UAS) of one yeast gene and downstream promoter elements including a functional TATA box of another yeast gene, for example a hybrid promoter including the UAS(s) of the yeast PH05 gene and downstream promoter elements including a functional TATA box of the yeast GAP gene (PH05-GAP hybrid promoter). A suitable constitutive PH05 promoter is e.g. a shortened acid phosphatase PH05 promoter devoid of the upstream regulatory elements (UAS) such as the PH05 (−173) promoter element starting at nucleotide −173 and ending at nucleotide −9 of the PH05 gene.

Gene transcription from vectors in mammalian hosts may be controlled by promoters derived from the genomes of viruses such as polyoma virus, adenovirus, fowlpox virus, bovine papilloma virus, avian sarcoma virus, cytomegalovirus (CMV), a retrovirus and Simian Virus 40 (SV40), from heterologous mammalian promoters such as the actin promoter or a very strong promoter, e.g. a ribosomal protein promoter, and from promoters normally associated with immunoglobulin sequences.

Transcription of a nucleic acid by higher eukaryotes may be increased by inserting an enhancer sequence into the vector. For example, the pEF-BOS vector (Mizushima, et al, 1990, Nucl. Acids Res. 18, 5322) contains the elongation factor 1α (EF-1α) promoter and enhancer which allows for elevated levels of expression of a cloned gene. Enhancers are relatively orientation and position independent. Many enhancer sequences are known from mammalian genes (e.g. elastase and globin). However, typically one will employ an enhancer from a eukaryotic cell virus. Examples include the SV40 enhancer on the late side of the replication origin (bp 100-270) and the CMV early promoter enhancer. The enhancer may be spliced into the vector at a position 5′ or 3′ to the desired nucleic acid, but is preferably located at a site 5′ from the promoter.

Advantageously, a eukaryotic expression vector may comprise a locus control region (LCR). LCRs are capable of directing high-level integration site independent expression of transgenes integrated into host cell chromatin, which is of importance especially where the gene is to be expressed in the context of a permanently-transfected eukaryotic cell line in which chromosomal integration of the vector has occurred.

Eukaryotic expression vectors will also contain sequences necessary for the termination of transcription and for stabilising the mRNA. Such sequences are commonly available from the 5′ and 3′ untranslated regions of eukaryotic or viral DNAs or cDNAs. These regions contain nucleotide segments transcribed as polyadenylated fragments in the untranslated portion of the mRNA encoding the immunoglobulin.

Particularly useful for practising the present invention are expression vectors that provide for the transient expression of nucleic acids in mammalian cells. Transient expression usually involves the use of an expression vector that is able to replicate efficiently in a host cell, such that the host cell accumulates many copies of the expression vector, and, in turn, synthesises high levels of the desired gene product.

Construction of vectors according to the invention may employ conventional ligation techniques. Isolated plasmids or DNA fragments are cleaved, tailored, and religated in the form desired to generate the plasmids required. If desired, analysis to confirm correct sequences in the constructed plasmids is performed in a known fashion. Suitable methods for constructing expression vectors, preparing in vitro transcripts, introducing DNA into host cells, and performing analyses for assessing gene product expression and function are known to those skilled in the art. Gene presence, amplification and/or expression may be measured in a sample directly, for example, by conventional Southern blotting, Northern blotting to quantitate the transcription of mRNA, dot blotting (DNA or RNA analysis), or in situ hybridisation, using an appropriately labelled probe which may be based on a sequence provided herein. Those skilled in the art will readily envisage how these methods may be modified, if desired.

The first and/or second reporters, immunoglobulins, peptide fragments etc may be directly introduced to the cell by microinjection, or delivery using vesicles such as liposomes which are capable of fusing with the cell membrane. Viral and other fusogenic peptides may also be used to promote membrane fusion and delivery to the cytoplasm of the cell.

Alternatively and preferably, the reporter(s) etc may be delivered into cells as protein fusions or conjugates with a protein capable of crossing the plasma membrane and/or the nuclear membrane. Preferably, the reporter(s) is fused or conjugated to a domain or sequence from such a protein responsible for the translocational activity. Preferred translocation domains and sequences include domains and sequences from the HIV-1-trans-activating protein (Tat), Drosophila Antennapedia homeodomain protein and the herpes simplex-1 virus VP22 protein.

Exogenously added HIV-1-trans-activating protein (Tat) can translocate through the plasma membrane and reach the nucleus to transactivate the viral genome. Translocational activity has been identified in amino acids 37-72 (Fawell et al., 1994, Proc. Natl. Acad. Sci. U. S. A. 91, 664-668), 37-62 (Anderson et al., 1993, Biochem. Biophys. Res. Commun. 194, 876-884) and 49-58 (having the basic sequence RKKRRQRRR) of HIV-Tat. Vives et al. (1997), J Biol Chem 272, 16010-7 identified a sequence consisting of amino acids 48-60 (CGRKKRRQRRRPPQC), which appears to be important for translocation, nuclear localisation and trans-activation of cellular genes. Intraperitoneal injection of a fusion protein consisting of β-galactosidase and a HIV-TAT protein transduction domain results in delivery of the biologically active fusion protein to all tissues in mice (Schwarze et al., 1999, Science 285, 1569-72)

The third helix of the Drosophila Antennapedia homeodomain protein has also been shown to possess similar properties (reviewed in Prochiantz, A., 1999, Ann NY Acad Sci, 886, 172-9). The domain responsible for translocation in Antennapedia has been localised to a 16 amino acid long peptide rich in basic amino acids having the sequence RQIKIWFQNRRMKWKK (Derossi, et al., 1994, J Biol Chem, 269, 10444-50). This peptide has been used to direct biologically active substances to the cytoplasm and nucleus of cells in culture (Theodore, et al., 1995, J Neurosci 15, 7158-7167). Cell internalisation of the third helix of the Antennapedia homeodomain appears to be receptor-independent, and it has been suggested that the translocation process involves direct interactions with membrane phospholipids (Derossi et al., 1996, J Biol Chem, 271, 18188-93). The VP22 tegument protein of herpes simplex virus is capable of intercellular transport, in which VP22 protein expressed in a subpopulation of cells spreads to other cells in the population (Elliot and O'Hare, 1997, Cell 88, 223-33). Fusion proteins consisting of GFP (Elliott and O'Hare, 1999, Gene Ther 6, 149-51), thymidine kinase protein (Dilber et al., 1999, Gene Ther 6, 12-21) or p53 (Phelan et al., 1998, Nat Biotechnol 16, 440-3) with VP22 have been targeted to cells in this manner.

Particular domains or sequences from proteins capable of translocation through the nuclear and/or plasma membranes may be identified by mutagenesis or deletion studies. Alternatively, synthetic or expressed peptides having candidate sequences may be linked to reporters and translocation assayed. For example, synthetic peptides may be conjugated to fluoroscein and translocation monitored by fluorescence microscopy by methods described in Vives et al. (1997), J Biol Chem 272, 16010-7. Alternatively, green fluorescent protein may be used as a reporter (Phelan et al., 1998, Nat Biotechnol 16, 440-3).

Any of the domains or sequences or as set out above or identified as having translocational activity may be used to direct the first and/or second reporter(s) into the cytoplasm or nucleus of a cell.

Generation of a Signal

In the methods of the present invention, a signal is advantageously generated by the interaction of two reporters, brought together by the binding of the reporters to the entity. The signal generated will thus be dependent on the nature of the molecules used in the methods of the invention.

In a first embodiment, the signal is the activation of apoptosis or programmed cell death in the cell. Stable interaction of two caspase 3 moieties disposed on each of the first and second reporters auto-activates caspase activity, which leads to the initiation of apoptosis in the cell. Apoptotic cell death is characterised by cellular shrinkage, chromatin condensation, cytoplasmic blebbing, increased membrane permeability and interchromosomal DNA cleavage (Kerr et al. (1992) FASEB J. 6:2450; and Cohen and Duke (1992) Ann. Rev. Immunol. 10:267), and any of these features may be used if necessary to detect the signal.

It will be appreciated that in the above embodiment, the signal may also be detected by assaying caspase activity in the cell.

In a separate embodiment, the signal may be generation of protease activity within the cell. Caspase activity may be detected this way, as described above. Furthermore, the protease activity may be an activity associated with ubiquitin-mediated proteolysis, preferably proteasome activity, most preferably 26S proteasome activity.

Ubiquitin-mediated proteolysis is reviewed in Ubiquitination and the Biology of the Cell (Edited by Jan-Michael Peters, J. Robin Harris and Dan Finley, Kluwer Academic Publishers, Dordrecht, the Netherlands). In ubiquitin-mediated proteolysis, proteins are targeted for degradation by covalent modification with ubiquitin. Three types of enzyme are involved in this task including ubiquitin-activating (E1), ubiquitin-conjugating (E2) and ubiquitin-ligating (E3) enzymes. Ubiquitin is a small and highly conserved protein that is covalently attached to a lysine residue on a target protein (to label it). The ubiquitin-associated enzymes are able to recognise damaged or misfolded proteins by changes in protein structure and amino acid sequences. A chain of ubiquitin molecules then forms at the attachment site of the target protein as the ubiquitin-conjugating enzyme adds additional ubiquitin molecules to the original one. The resulting polyubiquitin chain is recognised by proteasomes (the protein complexes at either end of the proteasome are involved in the recognition process) and the labelled protein is then degraded.

This embodiment may therefore be realised by the first reporter and the second reporter comprising domains of a component of the ubiquitin mediated proteolysis pathway, which, on stable interaction, generates an activity associated with that component and which leads to proteolysis. The component may comprise an ubiquitin-activating enzyme, a ubiquitin-conjugating enzyme or a ubiquitin-ligase (such as SCF). In a highly preferred embodiment of the invention, the first and second reporters comprise domains of a F-box protein or motif.

The F-box motif functions to mediate protein-protein interaction. F-box proteins were first described as components of SCF ubiquitin-ligase (E3) complexes (Skowyra et al., Cell 1997, 91:209-219; Feldman et al., Cell 1997, 91:221-230). SCF complexes contain four components: Skp1, a cullin, Rbx1/Roc1/Hrt1, and an F-box protein (Tyers and Jorgensen, Curr Opin Genet Dev 2000, 10:54-64; Deshaies, Annu Rev Cell Dev Biol 1999, 15:435-467; Craig and Tyers, Prog Biophys Mol Biol 1999, 72:299-328). SCF complexes facilitate interaction between substrates and ubiquitin-conjugating enzymes, which then covalently transfer ubiquitin onto substrates. Poly-ubiquitinated substrates are subsequently degraded by the 26S proteasome (Hershko and Ciechanover, Annu Rev Biochem 1998, 67:425-479). The F-box protein is the subunit of the SCF complex that binds specific substrates, and it links to the complex by binding Skp1 through the F-box itself. A detailed review of the F-box and its role in ubiquitin mediated proteolysis is provided by Kipreos and Pagano, 2000), Genome Biology, 1(5), reviews 3002, 1-3002.7). the F-box therefore has a crucial role in recruiting the proteosome for protein destruction.

The first and/or the second reporter may comprise an F-box motif. However, as the F-box motif comprises three helixes preferably the first reporter comprises one or more of the helixes, and the second reporter comprises the remaining helix or helices. Preferably, the first reporter comprises the first helix and the second reporter comprises the second and third helices; alternatively, the first reporter comprises the first and second helices while the second reporter comprises the third helices. Moreover, other portions of the F-box may be split, not necessarily to produce complete helices. Standard techniques known in the art may be used for construction of such reporters comprising helix domains or F-box portions.

Stable interaction between the first reporter and second reporter via binding to the entity enables association between the various helix domains and reconstitution of the F-box motif activity. This leads to ubiquitin labelling of the entity, or the polypeptide comprising the entity, and the destruction via proteolysis of the entity/polypeptide. As noted above, such proteolysis may be readily detected by means known in the art.

For example, F-box proteins such as TRCP may be cloned from mammalian and/or other cells or tissues and fused to scFv either directly, or via linkers such as Pro linkers (Pro₁₀) or Gly-Ser linkers ((Gly₄-Ser)₃). The scFv is advantageously located N-terminal in the construct. Constructs comprising both the F-box and the WD domain may also be employed. Where individual helices of the F-box are used, a first reporter advantageously comprises H1 or H1 & H2, and a second reported advantageously comprises H2 & H3 or H3, respectively. Details on the structure of the F-box are set forth in Schulman et al., (2000) Nature 408:381.

Any of the known F-box motifs may be used as a basis for this aspect of the invention. Furthermore, the F-box consensus sequence:

kpfpllrlpEeilrkilekldpidllRlrkvskkwrSlvdslniwfkFIe

may also be used. A list of F-box proteins, together with their sequence accessions, is provided in the table below.

| Proposed | Literature | Other | | Sequence |
|---|---|---|---|---|
| Gene Symbol | Symbol | Aliases | Organism | Accessions |
| SKP2 | FBL1 | FBXL1 | human | U33761 |
| FBXL2 | FBL2 | FBL3 | human | AF176518, |
| | | | | AF174589, |
| | | | | AF186273 |
| FBXL3A | FBL3A | | human | AF129532 |
| Fbxl3a | Fbl3a | | mouse | AF176521 |
| FBXL3B | FBL3B | | human | AF129533 |
| FBXL4 | FBL4 | FBL5 | human | AF174590, |
| | | | | AF176699, |
| | | | | AF199355 |
| FBXL5 | FBL5 | FBL4, | human | AF174591, |
| | | FLR1 | | AF176700, |
| | | | | AF199420, |
| | | | | AF142481 |
| FBXL6 | FBL6 | | human | AF174592 |
| Fbxl6 | Fbl6 | | mouse | AF176522 |
| FBXL7 | FBL7 | FBL6, | human | AF174593, |
| | | KIAA0840 | | AF199356 |
| Fbxl8 | Fbl8 | | mouse | AF176523 |
| FBXL9 | FBL9 | | human | AF176701 |
| Fbxl10 | Fbl10 | | mouse | AF176524 |
| FBXL11 | FBL11 | KIAA1004 | human | AB023221: |
| | | LILINA | | G4589652 |
| | | | | AF179221 |
| | | | | AL117517 |
| Fbxl11 | Fbl11 | | mouse | AI154332 |
| Fbxl12 | Fbl12 | | mouse | AF176525 |
| BTRC | FBW1A | betaTRCP1, | human | AF129530, |
| | | FWD1 | | Y14153 |
| FBXW1B | FBW1B | betaTRCP2, | human | AF176022 |
| | | KIAA0696 | | |
| FBXW2 | FBW2 | | human | AF176698, |
| | | | | AF129531 |
| Fbxw2 | Fbw2 | Fwd2, | mouse | AA145853, |
| | | MD6 | | AF140683, |
| | | | | X54352 |
| FBXW3 | FBW3 | | human | AF174606 |
| Fbxw4 | Fbw4 | | mouse | AF176519 |
| Fbxw5 | Fbw5 | | mouse | AF176520 |
| CCNF | FBX1 | FBXO1 | human | Z36714 |
| FBXO2 | FBX2 | | human | AF174594, |
| | | | | AF187318 |
| FBXO3 | FBX3 | | human | AF174595, |
| | | | | AF176702 |
| FBXO4 | FBX4 | | human | AF129534, |
| | | | | AF176703 |
| FBXO5 | FBX5 | | human | AF129535 |
| FBXO6 | FBX6 | FBG2 | human | AF129536 |
| Fbxo6a | Fbx6a | | mouse | AU067142 |
| Fbxo6b | Fbx6b | | mouse | AF176526 |
| FBXO7 | FBX7 | | human | AF129537 |
| FBXO8 | FBX8 | | human | AF174596 |
| Fbxo8 | Fbx8 | | mouse | AF176527 |
| FBXO9 | FBX9 | NY-REN-57 | human | AF174597, |
| | | | | AF176704 |
| FBXO10 | FBX10 | | human | AF174598, |
| | | | | AF176705 |
| FBXO11 | FBX11 | | human | AF174599, |
| | | | | AF176706 |
| Fbxo12 | Fbx12 | | mouse | AF176528 |
| Fbxo13 | Fbx13 | | mouse | AF176529 |
| Fbxo14 | Fbx14 | | mouse | AU066822 |
| Fbxo15 | Fbx15 | | mouse | AF176530 |
| Fbxo16 | Fbx16 | | mouse | AF176531 |
| Fbxo17 | Fbx17 | | mouse | AF176532 |
| Fbxo18 | Fbx18 | | mouse | AF184275 |
| Fbxo19 | Fbx19 | | mouse | AA501293 |
| FBXO20 | FBX20 | | human | AF174600 |
| FBXO21 | FBX21 | KIAA0875 | human | AF174601 |
| FBXO22 | FBX22 | | human | AF174602 |
| FBXO23 | FBX23 | | human | AF174603 |
| FBXO24 | FBX24 | | human | AF174604 |
| FBXO25 | FBX25 | | human | AF174605 |
| FBXO29 | FBX29 | | human | AF176707 |
| Fbxo30 | Fbx30 | | mouse | AI836688 |

Preferably, the F-box domain is placed at the C-terminus of a reporter; thus, in a highly preferred embodiment of the invention, a reporter comprises an scFv fused N—C to a F-box domain.

In a second embodiment, the signal is the emission or absorption of electromagnetic radiation, and the signal-generation molecules may be fluorophores. Particularly preferred are fluorescent molecules which participate in energy transfer (FRET).

FRET is detectable when two fluorescent labels which fluoresce at different frequencies are sufficiently close to each other that energy is able to be transferred from one label to the other. FRET is widely known in the art (for a review, see Matyus, 1992, J. Photochem. Photobiol. B: Biol., 12: 323-337, which is herein incorporated by reference). FRET is a radiationless process in which energy is transferred from an excited donor molecule to an acceptor molecule; the efficiency of this transfer is dependent upon the distance between the donor an acceptor molecules, as described below. Since the rate of energy transfer is inversely proportional to the sixth power of the distance between the donor and acceptor, the energy transfer efficiency is extremely sensitive to distance changes. Energy transfer is said to occur with detectable efficiency in the 1-10 nm distance range, but is typically 4-6 nm for favourable pairs of donor and acceptor.

Accordingly, the invention may be practised by choosing suitable pairs of donor and acceptor molecules, so that one of the first and second reporters comprises a donor molecule and the other of the first and second reporters comprises an acceptor molecule. When the reporters bind to the entity, the donor molecule and the acceptor molecule are brought together so that energy transfer occurs.

Radiationless energy transfer is based on the biophysical properties of fluorophores. These principles are reviewed elsewhere (Lakowicz, 1983, Principles of Fluorescence Spectroscopy, Plenum Press, New York; Jovin and Jovin, 1989, Cell Structure and Function by Microspectrofluorometry, eds. E. Kohen and J. G. Hirschberg, Academic Press, both of which are incorporated herein by reference). Briefly, a fluorophore absorbs light energy at a characteristic wavelength. This wavelength is also known as the excitation wavelength. The energy absorbed by a fluorochrome is subsequently released through various pathways, one being emission of photons to produce fluorescence. The wavelength of light being emitted is known as the emission wavelength and is an inherent characteristic of a particular fluorophore. Radiationless energy transfer is the quantum-mechanical process by which the energy of the excited state of one fluorophore is transferred without actual photon emission to a second fluorophore. That energy may then be subsequently released at the emission wavelength of the second fluorophore. The first fluorophore is generally termed the donor (D) and has an excited state of higher energy than that of the second fluorophore, termed the acceptor (A). The essential features of the process are that the emission spectrum of the donor overlap with the excitation spectrum of the acceptor, and that the donor and acceptor be sufficiently close. The distance over which radiationless energy transfer is effective depends on many factors including the fluorescence quantum efficiency of the donor, the extinction coefficient of the acceptor, the degree of overlap of their respective spectra, the refractive index of the medium, and the relative orientation of the transition moments of the two fluorophores. In addition to having an optimum emission range overlapping the excitation wavelength of the other fluorophore, the distance between D and A must be sufficiently small to allow the radiationless transfer of energy between the fluorophores.

In a FRET assay, the fluorescent molecules are chosen such that the excitation spectrum of one of the molecules (the acceptor molecule) overlaps with the emission spectrum of the excited fluorescent molecule (the donor molecule). The donor molecule is excited by light of appropriate intensity within the donor's excitation spectrum. The donor then emits some of the absorbed energy as fluorescent light and dissipates some of the energy by FRET to the acceptor fluorescent molecule. The fluorescent energy it produces is quenched by the acceptor fluorescent molecule. FRET can be manifested as a reduction in the intensity of the fluorescent signal from the donor, reduction in the lifetime of its excited state, and re-emission of fluorescent light at the longer wavelengths (lower energies) characteristic of the acceptor. When the donor and acceptor molecules become spatially separated, FRET is diminished or eliminated.

Suitable fluorophores are known in the art, and include chemical fluorophores and fluorescent polypeptides, such as GFP and mutants thereof which fluoresce with different wavelengths or intensities (see WO 97/28261). Chemical fluorophores may be attached to immunoglobulin by incorporating binding sites therefor into the immunoglobulin during the synthesis thereof.

Preferably, however, the fluorophore is a fluorescent protein, which is advantageously GFP or a mutant thereof. GFP and its mutants may be synthesised together with the binding means (where this is a polypeptide such as an immunoglobulin) by expression therewith as a fusion polypeptide, according to methods well known in the art. For example, a transcription unit may be constructed as an in-frame fusion of the desired GFP and the immunoglobulin, and inserted into a vector as described above, using conventional PCR cloning and ligation techniques.

In a third embodiment, the binding domain is linked to a association domain, the association domain being capable of giving rise to a biological signal. Preferred association domains are polypeptide molecules, which advantageously interact to form a transcription factor, or another regulatory molecule, which modulates gene expression within the cell.

Exemplary transcription factor molecules have been described in the literature, for example by Fields & Song, (1989) Nature 340:245-246, which is incorporated herein by reference. In a preferred embodiment, the immunoglobulin molecule is expressed as fusion protein with the activation domain of the HSV1 VP16 molecule. This transcription factor domain is capable of upregulating gene transcription from a promoter to which it is bound through a DNA binding activity. The latter is provided by the DNA-binding domain of the E. coli LexA polypeptide, which is expressed as a fusion protein with an immunoglobulin polypeptide.

The biological signal may be any detectable signal, such as the induction of the expression of a detectable gene product. Examples of detectable gene products include bioluminescent polypeptides such as luciferase, fluorescent polypeptides such as GFP, polypeptides detectable by specific assays, such as β-galactosidase and CAT, and polypeptides which modulate the growth characteristics of the host cell, such as enzymes required for metabolism such as HIS3, or antibiotic resistance genes such as G418. In a preferred aspect of the invention, the signal is detectable at the cell surface. For example, the signal may be a luminescent or fluorescent signal, which is detectable from outside the cell and allows cell sorting by FACS or other optical sorting techniques. Alternatively, the signal may comprise the expression of a cell surface marker, such as a CD molecule, for example CD4 or CD8, which may itself be labelled, for example with a fluorescent group, or may be detectable using a labelled antibody. Use of optical sorting, such as FACS, enables a collection of cells to be panned and selects for cells which contain the entity.

The invention is further described, for the purposes of illustration only, in the following examples.

### EXAMPLES

### Example 1

### Construction of Expression Plasmids

pM-βgal, pNL-scFvR4-VP16, pNL-scFvF8-VP16 and pNL-scFv-IN33-VP16 have been described previously (Visintin et al., 1999, Proc. Natl. Acad. Sci. USA 96, 11723-11728). pRSV-Luc (Firefly luciferase expression vector) has also been described previously (de Wet, J. R., 1987, Mol Cell Biol 7, 725-37) and pEGFP-N1 (enhanced green fluorescence protein, GFP expression vector) is commercially available from CLONTECH Laboratories, Inc. (Palo Alto, Calif., USA).

The pEF-βgal (β-galactosidase expression vector) is created by subcloning the coding sequence of β-galactosidase and SV40 polyA from pBSpt-βgal (Greenberg, et al, 1990, Nature 344, 158-160) into the pEF-BOS mammalian expression vector (Mizushima, et al, 1990, Nucl. Acids Res. 18, 5322).

The shuttle vectors pBS-R4 and pBS-F8 are made by cloning the ClaI-EcoRI fragment of pNL-scFvR4-VP16 and pNL-scFvF8-VP16 respectively into pBSpt

The pEF-R4-DBD (scFvR4-GAL4DBD fusion expression vector) is constructed as follows. The Gal4 DNA binding domain sequence (PCR amplified from pGALO, Dang, et al., 1991, Mol. Cell. Biol. 11, 954-962) is cloned in-frame with the scFvR4 in the EcoRI site of pBS-R4. pEF-R4-DBD is made by cloning the ClaI-SpeI fragment of R4-Gal4 DNA binding domain fusion into pEF-BOS.

The pEF-R4-CP3 and pEF-F8-CP3 (scFvR4 and scFvF8-caspase 3 fusion expression vectors): The human caspase 3 sequence has been deposited as GENBANK accession numbers U26943 and U13737. The human caspase 3 sequence is PCR amplified from a cDNA clone (a gift from Dr. Marion MacFarlane) and subcloned as an EcoRI-SpeI fragment in-frame at the 3′ end of the scFv sequence in both pBS-R4 and pBS-F8. The ClaI-SpeI fragments of the scFv-caspase 3 fusion are cloned into pEF-BOS to give pEF-R4-CP3 and pEF-F8-CP3 respectively.

The pEF-R4-CP3(C163S) (scFvR4-caspase3 mutant fusion expression vector) is made by mutating the cysteine (TGC) at position 163 of wild type caspase 3 into a serine (TCC) by site directed mutagenesis using the QuickChange™ Site-Directed Mutagenesis Kit (Stratagene) according to the manufacturer's instructions.

The pEF-IN33-CP3 and pEF-IN33-CP3(C163S) (scFvIN33-caspase3 and mutant expression vectors): XhoI-EcoRI fragment of pNL-scFvIN33-VP16 is cloned into the vector backbones of pBS-R4-CP3 and pBS-R4-CP3(C163S) digested with XhoI and EcoRI to create pBS-IN33-CP3 and pBS-IN33-CP3(C163S) respectively. pEF-IN33-CP3 and pEF-IN33-CP3(C163S) are constructed by cloning the XhoI-SpeI fragment of pBS-IN33-CP3 and pBS-IN33-CP3(C163S) into pEFBOS.

pEF-HIVIN-βgal (HIV integrase (a.a.259-288)-βgalactosidase fusion expression vector): NcoI-SalI fragment of pBlueScript-βgal is subcloned into pEF/myc/cyto (Invitrogen) and the PCR amplified HIV-1 integrase epitope (amino acid no. 259-288) is cloned as a NcoI fragment in-frame at the 5′ end of the β-gal sequence to give pEF-HIVIN-βgal.

### Example 2

### Mammalian Cell Culture and Transfection

Chinese hamster ovary (CHO) cells are grown in α minimal essential medium (GIBCO BRL) with 10% foetal calf serum, penicillin and streptomycin. 2×10⁵ CHO cells are seeded onto a 35 mm petri-dish 16-24 hours before transfection. Transfection is performed using Lipofectamine® (GIBCO BRL) according to the manufacturer's instructions with 500 ng of pEF-βgal, pEF-HIVIN-βgal and pRSV-Luc, 50 ng of pEGFP-N1 and 250 ng of pEF-scFv-CP3/CP3(C163S) except for pEF-IN33-CP3/CP3(C163S) for which 50 ng is used. Cells are harvested 60 hours after transfection or for the time-course experiment, at 12, 24, 36, 48 and 60 hours for further analyses.

CHO-CD4 line is a gift from Dr. C.V. Dang and has been reported previously (Fearon, et al, 1992, Proc. Natl. Acad. Sci. USA 89, 7958-7962). It is maintained with a minimal essential medium, 10% foetal calf serum and 1 mg/ml G418 (GIBCO BRL). Lipofectamine® transfection of CHO/CD4 cells growing on 100 mm dishes at 50-60% confluence is performed using 5 μg of each plasmid unless stated otherwise.

### Example 3

### FACS Analyses for CD4 Expression

48 hours after transfection, transfected CHO-CD4 cells are detached with Cell Dissociation Solution (Sigma) and are made into single cell suspension in PBS. CD4 expression is analysed by binding a mouse anti-human CD4 antibody (Pharmingen) at 1:50 dilution and a second antiserum goat anti-mouse polyclonal antibody labelled with a fluorescein isothiocyanate (FITC) (Pharmingen) at 1:100 dilution. The relative fluorescence of the cells is measured with a FACSCalibur (Becton Dickinson) and the data are further analysed by the software CELLQuest (Becton Dickinson).

### Example 4

### Western Blotting

CHO cells are transfected with scFvR4-caspase3, scFvR4-caspase3(C163S) and scFvF8-caspase3 expression vectors. 48 hours after transfection, cells are lysed with buffer containing 10 mM Hepes pH 7.6, 250 mM NaCl, 5 mM EDTA and 0.5% Nonidet P40. The lysates are fractionated by 12% SDS-PAGE and transferred to nitrocellulose membrane. The membrane is incubated with anti-human caspase3 antibody (Santa Cruz Biotechnology) and the secondary HRP conjugated anti-goat antibody (Santa Cruz Biotechnology). Detection is performed with ECL Western blotting detection reagents (Amersham).

### Example 5

### β-Galactosidase and Luciferase Activities Assays

Transfected CHO cells are lysed using 300 μl Reporter Lysis Buffer (Promega) per 35 mm petri-dish at the specified time points after transfection. β-galactosidase activities are measured using β-galactosidase Enzyme Assay System (Promega) according to the manufacturer's instructions. Luciferase activity measurements are performed using Luciferase Assay System (Promega) and a luminometer. Two separate independent transfection are done and the averaged result is presented.

### Example 6

### Apoptosis Assay

CHO cells are co-transfected with green fluorescent protein expression vector pEGFP-N1, the various scFv fusion expression vectors, and with or without pEF-βgal. 36 hours after transfection, GFP-positive CHO cells are sorted using a flow cytometer. Genomic DNA is extracted from approximately 5000 cells. The presence or absence of nuclear DNA products is detected using the ApoAlert LM-PCR Ladder Assay Kit (Clontech). Genomic DNA is amplified using the ApoAlert primers which amplify DNA generated from the chromatin beads resulting from apoptosis. PCR products are visualised by ethidium bromide staining after fractionation on 1.5% agarose gels. As a control for DNA yield in each set of FACS sorted cells, primers specific for Chinese Hamster actin are used (5′GGCGTGATGGTGGGCATGGGCCAG3′ and 5′CTGGTCATCTTTTCACGGTTGGC3′) for PCR. The PCR reaction consisted of 35 cycles of 94° c. denaturing for 1 minute, 65° c. annealing for 1 minute and 72° c. extension for 1 minute. The products were also visualised on 1.5% agarose gels.

### Example 7

### Activation of Transcription by Proximity of Anti-β-Galactosidase ScFv-VP16 Bound to Antigen

The crystal structure of β-galactosidase is known, showing that a tetramer is necessary for enzyme activity (Jacobson et al, 1994, Nature 369, 761-766). Therefore antibodies which bind to β-galactosidase may do so at four separate antigenic sites per active enzyme. An anti-β-galactosidase scFv has been described, scFv-R4, which binds to β-galactosidase in both bacterial (Martineau, et al., 1998, J Mol Biol 280, 117-127) and mammalian cells (Visintin, et al., 1999, Proc. Natl. Acad. Sci. USA 96, 11723-11728) but does not affect β-galactosidase function. This model intracellular antibody system is used to determine if proximity of scFv antibody fragments in vivo would cause measurable biochemical effects. For this assessment, we initially determine if transcriptional transactivation could be mediated by scFv-R4 binding to antigen. Our assay consists of co-expression of β-galactosidase with scFv-R4 fused to a Gal4 DNA-binding domain (DBD) and scFV-R4 fused to the VP16 transcriptional transactivation domain, in a CHO cell line with a CD4 reporter gene controlled by the GAL4 promoter (Fearon, et al., 1992, Proc. Natl. Acad. Sci. USA 89, 7958-7962). The CHO-CD4 cells express CD4 on their cell surface if the reporter gene is activated. Therefore, if the intracellular antibody scFv-R4-DBD and scFv-R4-VP16 bind to β-galactosidase in CHO-CD4 cells, a transcription complex should be created which causes activation of the CD4 gene (illustrated in FIG. 1A).

Various combinations of plasmids are transfected in CHO-CD4 cells and, 60 hours after transfection, cell surface CD4 is measured by FACS analyses (FIG. 1B). When β-galactosidase itself is directly linked to the GAL4-DBD, expression of scFv-R4-VP16 fusions results in efficient CD4 surface expression (panel 2) whereas co-expression of scFv-R4 fused to GAL4 DBD or to VP16 in the absence of β-galactosidase antigen fails to activate CD4 (panel 3). Therefore the DBD-β-galactosidase fusion can interact with the DNA-binding sites of the CD4 reporter and a transcription complex is created. scFv-R4, linked to VP16 activation domain also binds to the β-galactosidase epitopes on the DBD-β-galactosidase tetramer.

Next, β-galactosidase is co-expressed with scFv-R4 fused to the DNA-binding domain or fused to the VP16 activation domain to assess the formation of a DNA-binding complex. For this to be effective, both scFv-DBD and scFv-VP16 must bind to different sites on the same galactosidase tetramer. We therefore titrated different amounts of expression vectors to alter the ratio of scFv-DBD and scFv-VP16 fusion proteins (FIG. 1B panels 4-9). CD4 reporter gene activation is observed in all cases but less efficiently than the DBD-β-galactosidase co-expression (the percentage of cells expressing CD4 in these transient assays ranging from 0.24% to 2.8%). The relative inefficiency presumably reflects the bulkiness of the protein complex in the transcription assay and the need for multiple binding sites on each β-galactosidase. The degree of CD4 expression is dependent on the ratio between scFvR4-VP16 and scFvR4-DBD (FIG. 1B panels 6, 7 and 8) as expected since the two different scFvR4 fusion proteins compete for the same binding sites on the β-galactosidase tetramer. It is confirmed that no detectable dimerisation occurs between scFvR4 since CD4 activation does not occur when the scFvR4-VP16 and scFvR4-DBD are expressed in the absence of β-galactosidase (FIG. 1B panel 3). We conclude that protein domains that are linked to scFvR4 can be brought into sufficiently close proximity for biochemical interactions when the scFvR4 binds to β-galactosidase epitopes in vivo.

The above experiments show that it is possible to induce a cell to generate a detectable signal using the method according to the first aspect of the invention. Moreover, the method according to the second aspect of the invention may be used to detect an entity comprising an intracellular protein, by detection of a signal consisting of transcriptional activity. The transcriptional activity is generated by the stable interaction of a VP16 activation domain provided on a first reporter with a GAL4 DBD provided on a second reporter, the stable interaction arising through binding of the first and second reporters to their targets on the intracellular protein.

### Example 8

### ScFv-Caspase 3 Fusion Causes Apoptosis After Binding to Antigen

If the necessary components of a transcription complex can be brought close enough on the β-galactosidase tetramer to give activity, it suggested that the molecular distance between the scFv-R4 antigenic sites might be small enough to bring the caspase 3 moieties close enough together, to cause auto-activation and triggering of apoptosis (as illustrated in FIG. 2A).

An assessment of this is made by co-transfecting CHO cells with the reporter β-galactosidase expression plasmid along with various scFv-R4 expression clones, including one encoding a fusion of scFvR4 with caspase 3. Expression of each of the scFv fusion proteins is confirmed using anti-caspase3 antibody in Westerns blots of protein extracts from CHO cells transfected with the expression vectors (FIG. 2B). 60 hours after transfection, the cells are assayed for β-galactosidase activity (FIG. 2C).

While β-galactosidase is detected in the control transfection (panel 1), when the scFV-R4 is linked to caspase 3, however, we observe very little β-galactosidase activity level (panel 3). This loss of β-galactosidase activity is dependent on the activity of caspase 3, as judged by the effect of an inactivating caspase 3 mutation (MacCorkle, et al., 1998, Proc Natl Acad Sci U S A 95, 3655-60) in which the catalytic cysteine is mutated to a serine (C163S, panel 4). In addition, no significant difference is observed when the β-galactosidase reporter is co-expressed with scFv-R4 fused to VP16 only (panel 2). The antibody specificity causing the lack of β-galactosidase activity is shown by co-transfecting a non-specific scFv (scFv-F8, Tavladoraki, et al., 1993, Nature 366, 469-472) fused with caspase 3. This combination has no effect on β-galactosidase levels. These data therefore suggest that the reduction in β-galactosidase activity when scFv-R4-caspapse 3 is co-transfected is not due to a neutralising effect on β-galactosidase. Rather, it is due to the proteolytic activity of the activated caspase 3 causing apoptosis of the transfected cells. It is also of note that scFv-caspase 3 fusion alone is not toxic to cells (panel 5) which is consistent with previous reports (MacCorkle, et al., 1998, Proc Natl Acad Sci U S A 95, 3655-60; Fan, et al., 1999, Hum Gene Ther 10, 2273-85; Yoshioka, et al, 1999, Gene Ther 6, 1952-9).

### Example 9

### Luciferase Assay for Apoptosis Induced by ScFv-Caspase 3 Fusion

Induction of cell killing by the scFvR4-caspase 3 fusion molecule, dependent on the interaction of specific scFvR4-caspase 3 and β-galactosidase antigen, is confirmed using an independent reporter to which the scFvR4 antibody does not bind.

CHO cells are transfected with the reporter pRSV-Luc, constitutively expressing Firefly luciferase, together with either scFvR4-caspase 3, scFvR4-VP16, mutant scFvR4-caspase 3(C163S) or scFvF8-caspase 3 in the presence or absence of β-galactosidase expression (i.e. scFv-R4 antigen). Luciferase activity is measured 60 hours after transfection, as a measure of cell viability in the presence of scFv fusion proteins (FIG. 3A).

These data show about 80% decrease in luciferase activity when scFvR4-caspase 3 is expressed along with β-galactosidase (panel 3, front row). However, this lack of β-galactosidase activity in cells transfected with scFv-R4-caspase 3 would appear to be due to antigen-dependent cell death, rather than toxicity of the expressed scFv-R4-caspase 3 alone, as no loss of viability is observed when scFv-R4-caspase 3 is expressed with an independent reporter without β-galactosidase (panel 3, back row). Caspase-dependence is demonstrated by transfection with a construct expressing the scFvR4-caspase mutant protein fusion, with the transfected cells showing no reduction in luciferase activity, either in the presence and absence of β-galactosidase expression. Finally, antibody-specificity is confirmed using the non-specific scFv-F8 which does not affect reporter gene activity.

A time-course of luciferase activity is performed with transfected CHO cells, assayed at various times after transfection with or without the β-galactosidase vector (respectively FIGS. 3B and 3C). In cells expressing both scFvR4-caspase 3 and β-galactosidase, the luciferase level increases slowly in the first 36 hours to a peak which is lower than the corresponding level in the two controls expressing scFvR4-caspase3(C163S) or scFvF8-caspase3 and β-galactosidase (FIG. 3B). Furthermore, unlike the two controls in which luciferase levels continue to rise, there is a drop in the enzyme level in cells co-transfected with scFvR4-caspase3 and β-galactosidase. On the other hand, in the absence of β-galactosidase, scFvR4-caspase 3 does not affect luciferase level and is comparable to the scFvR4-caspase 3(C163S) and scFvF8-caspase 3 (FIG. 3C). These results indicate that induced apoptosis is antibody and antigen specific, and dependent on caspase 3 activity.

### Example 10

### Apoptosis Induced by ScFv-Caspase 3 Fusion Assayed by Chromatin Degradation

Our results previous results with the β-galactosidase and luciferase assays show that apoptosis is dependent on active caspase 3, and that scFv-caspase 3 causes apoptosis after binding to antigen. These results are confirmed by assaying transfected cells for the presence of a chromatin bead ladder. The presence of a chromatin bead ladder is a hallmark of apoptotic cell death, and is caused by nuclease digestion of chromatin (Wyllie, et al., 1980, Nature 284, 555-6).

CHO cells are transfected with a marker plasmid, pEGFP-N1, which expresses green fluorescent protein (GFP), together with those expressing the various scFv fusion proteins, and with or without the β-galactosidase expression vector. After 36 hours, transfected cells which express GFP (therefore also scFv and β-galactosidase) are enriched by selection using a fluorescence activated cell sorter. Genomic DNA is extracted from the sorted cells. DNA fragments emanating from the apoptosis-mediated chromatin digestion are assessed by the ligation-mediated PCR procedure (Staley, et al., 1996 Cell Death & Differen. 4, 66-75) (FIG. 4).

We only find evidence of chromatin beads in the DNA prepared from cells co-transfected with scFvR4-caspase 3 and β-galactosidase (lane 2) and not in those transfected with β-galactosidase and scFvR4-VP16 (lane 1), scFvR4-caspase3(C163S) (lane 3) or scFvF8-caspase 3 (lane 4). Yields of DNA in each are comparable as determined by PCR using actin gene primers (FIG. 4B). Moreover, scFvR4-caspase 3 transfected in the absence of β-galactosidase did not generate the DNA ladder (lane 5), indicating that the apoptosis is dependent on the specific interaction between the intracellular antibody-caspase fusion (scFv-R4-caspase 3) and antigen (β-galactosidase).

We have therefore shown that it is possible to induce apoptosis within a cell by making use of first and second reporters each comprising an antibody which binds to an intracellular target and a caspase 3 molecule. The method according to the third aspect of our invention may therefore be used to kill any cell which comprises an entity.

### Example 11

### General Applicability of Intracellular Antibody Mediated Apoptosis

To consolidate the general applicability of the intracellular antibody mediated apoptosis approach, a second system is developed using a different antigen and intracellular antibody pair, namely a small antigenic epitope of HIV-1 integrase and specific antibody recognising this epitope in vivo (scFvIN33; Levy-Mintz, et al., 1996, J. Virol. 70, 8821-8832).

The structure of β-galactosidase is known (Jacobson, et al., 1994, Nature 369, 761-766). A prediction from this structure is that any protein linked to the N-terminus of β-galactosidase monomer will be positioned at the interface of the tetrameric β-galactosidase molecule. As a result, the physical distance between the linked moieties is expected to be short, as will the distance between the specific antibodies that bind to them. Therefore, an expression construct, pEF-HIVIN-β-gal, is made in which the HIV-1 integrase amino acids 259-288 (i.e., those recognised by scFvIN33; Bizub-Bender, et al., 1994, AIDS Res Hum Retroviruses 10, 1105-15) are fused at the N-terminus of β-galactosidase. This construct is co-expressed in CHO cells with an scFvIN33-caspase 3 fusion protein. Interaction of antibody and antigen should cause caspase-mediated apoptosis as depicted in FIG. 5A.

60 hours after transfection, β-galactosidase activity is assayed to determine cell viability. In parallel with our findings with anti-β-galactosidase scFv, the expression of β-galactosidase is markedly decreased when the HIV-integrase-β-galactosidase fusion is co-expressed with scFvIN33-caspase 3 (FIG. 5A, panel 2, front row) compared with the HIV-integrase-β-galactosidase fusion alone (panel 1, front row). Furthermore, expression of scFvIN33-caspase 3 with wild type β-galactosidase did not result in significant reduction in β-galactosidase activity (FIG. 5 panel 2, back row) indicating that cell death occurs in response to dimerisation of scFv-caspase 3 after binding to sites on the HIV-integrase-β-galactosidase fusion (as illustrated in FIG. 5A) and is not due to auto-toxicity of scFvIN33-caspase 3.

The requirement for active caspase 3 is demonstrated by using a mutant scFv-caspase, scFvIN33-caspase 3(C163S) (panel 3) and the antibody specificity is shown using the non-specific antibody scFvF8-caspase 3 (panel 5). Neither of these fusion proteins is seen to affect β-galactosidase levels. On the other hand, the anti-μ-galactosidase antibody fusion, scFvR4-caspase 3, causes cell death in cells expressing wild type β-galactosidase and HIV integrase β-galactosidase fusion as expected (panel 4, back and front rows). Cell death in this model system is, therefore, antigen specific, antibody specific and dependent on active caspase 3.

### Example 12

### Detection of a Cell by Production of a Fluorescence Signal

The presence of an entity within a cell may be determined by utilising a fusion protein consisting of an antibody linked to a fluorophore, and monitoring a fluorescence signal. Two constructs are made, one expressing a fusion protein consisting of scFv-R4 fused with Yellow Fluorescent Protein (YFP), and the other expressing a fusion protein consisting of scFv-R4 fused with Cyan Fluorescent Protein (CFP). These constructs are made by subcloning a YPF coding fragment from pYFP-N1 or a CFP coding fragment from pCFP-N1 (CLONTECH Laboratories) into pNL-scFvR4.

The constructs are transfected into CHO cells together with a pEF-βgal plasmid expressing β-galactosidase. As controls, CHO cells are also transfected with the construct expressing scFv-R4-YFP and the construct expressing scFv-R4-CFP only but without pEF-βgal plasmid. Further controls include transfection with co-transfecting non-specific scFvs fused with CFP and YFP, with or without the pEF-βgal plasmid.

60 hours after transfection, cells are subjected to FACS sorting. A CFP/YFP FRET filter set (Omega Optical Incorporated) is used to distinguish FRET signals from ordinary fluorescent signals. The cells which are transfected with the plasmids expressing β-galactosidase, the scFv-R4-CFP fusion and the scFv-R4-YFP are found to exhibit FRET, while the cells which are transfected with the scFv-R4-CFP fusion and the scFv-R4-YFP only (without β-galactosidase) do not exhibit FRET. Furthermore, cells which are transfected with scFv-F8-CFP and scFv-F8-YFP expressing constructs (with or without a construct expressing β-galactosidase) are found not to exhibit FRET.

We have shown that when two or more scFv-caspase 3 fusion proteins bind to the epitopes of an antigen that are close together, the caspase 3 moieties can be activated and trigger apoptosis. In this way, cells which express a target antigen are selectively killed. The validity of this process has been demonstrated with two pairs of model antibodies, namely anti-β-galactosidase scFvR4-caspase 3 fusion and anti-HIV integrase scFvIN33-caspase fusion. Since β-galactosidase exists as tetrameric form under physiological conditions (Jacobson et al, 1994, Nature 369, 761-766), there are four binding sites for the scFvR4 in the tetramer. When scFvR4 is linked at the N-terminus of caspase 3, the binding of the scFvR4-caspase3 fusion proteins to the tetrameric β-galactosidase can bring the linked caspase 3 moieties to close proximity. This results in activation of caspase 3 and leads to apoptosis. In the second model using an HIV integrase epitope linked to β-galactosidase, we provide further proof of triggering apoptosis by the specific binding between intracellular antibody-caspase 3 fusion proteins and the respective antigen. In each case, the cells specifically expressing target antigen are killed, demonstrating the general applicability and specificity of intracellular antibody-mediated cell killing.

Each of the applications and patents mentioned above, and each document cited or referenced in each of the foregoing applications and patents, including during the prosecution of each of the foregoing applications and patents (“application cited documents”) and any manufacturer's instructions or catalogues for any products cited or mentioned in each of the foregoing applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A method of inducing a cell to generate a detectable signal, the method comprising the steps of:
(a) providing a cell comprising an entity;
(b) providing a first reporter and a second reporter, in which a stable interaction of the first reporter with the second reporter leads to generation of a detectable signal; and
(c) allowing the first reporter and the second reporter to bind to the entity, such that binding of the reporters to the entity leads to stable interaction of the first reporter with the second reporter and generation of a signal.

2. A method of detecting an entity within a cell, the method comprising the steps of:
(a) providing a first reporter and a second reporter, in which a stable interaction of the first reporter with the second reporter leads to generation of a detectable signal;
(b) allowing the first reporter and the second reporter to bind to the entity, such that binding of the reporters to the entity leads to stable interaction of the first reporter with the second reporter and generation of a signal; and
(c) detecting the entity by monitoring the signal.

3. A method according to claim 1 or 2, in which the signal is the activation of a cell killing mechanism.

4. A method according to claim 3, in which the cell killing mechanism is apoptosis.

5. A method according to any preceding claim, in which the signal is generation of a cysteine protease activity.

6. A method according to claim 5, in which the cysteine protease activity is a caspase activity.

7. A method according to any preceding claim, in which the first reporter and the second reporter each comprise a caspase molecule selected from caspase 3 and caspase 8.

8. A method according to claim 7, in which the binding of the reporters to the entity leads to auto-activation of the caspase molecules and activation of apoptosis in the cell.

9. A method according to claim 8, in which the caspase molecule is caspase 3.

10. A method according to claim 1 or 2, in which the signal is generation of a transcriptional activity.

11. A method according to claim 10, in which the first reporter and the second reporter comprise domains of a transcription factor.

12. A method according to claim 10 or 11, in which either the first reporter or the second reporter comprises the DNA binding domain (DBD) of Gal4, and the other of the first reporter and the second reporter comprises a VP16 activation domain.

13. A method according to claim 10, 11 or 12, in which the signal is detected by monitoring the expression of a reporter gene.

14. A method according to claim 13, in which the reporter gene is CD4.

15. A method according to claim 1 or 2, in which the signal is the generation of a luminescence inducing activity.

16. A method according to claim 1 or 2 in which the signal is a fluorescent signal.

17. A method according to claim 16, in which the fluorescent signal is emitted by fluorescein isothiocyanate, rhodamine, Green Fluorescent Protein, Cyan Fluorescent Protein, Yellow Fluorescent Protein, Blue Fluorescent Protein or Red Fluorescent Protein.

18. A method according to claim 16 or 17, in which the fluorescent signal is modulated by fluorescent resonance energy transfer (FRET).

19. A method according to any preceding claim, wherein one or both of the first reporter and the second reporter comprises a target specific binding polypeptide or nucleic acid aptamer.

20. A method according to any one of claims 1 to 18, in which one or both of the first reporter and the second reporter comprises an immunoglobulin.

21. A method of killing a cell, the method comprising the steps of:
(a) providing a cell comprising an entity;
(b) providing a first reporter comprising a first immunoglobulin and a second reporter comprising a second immunoglobulin;
(c) providing a first caspase molecule linked to the first immunoglobulin and a second caspase molecule linked to the second immunoglobulin, the first and the second caspase molecules being capable of stably interacting to generate a caspase activity to cause apoptosis in the cell; and
(d) allowing the first immunoglobulin and the second immunoglobulin to bind to the entity, such that binding of the reporters to the entity leads to stable interaction of the caspase molecules to generate caspase activity and apoptosis in the cell.

22. A method according to claim 21, in which the caspase is caspase 3.

23. A method according to claim 20, 21 or 22, in which the immunoglobulin is an antibody, a T-cell receptor, or a fragment thereof.

24. A method according to any of claims 20 to 23, in which the immunoglobulin is an antibody selected from an Fv, a single chain Fv (scFv), a Fab or a F(ab′)₂.

25. A method according to any of claims 20 to 24, in which the immunoglobulin is an intracellular single chain Fv.

26. A method according to any of claims 20 to 25, in which the entity comprises an epitope recognised by the immunoglobulin.

27. A method according to any of claims 20 to 26, in which the immunoglobulin is provided by expression of nucleic acid within the cell.

28. A method according to claim 27, in which the nucleic acid is obtained from a phage library encoding a repertoire of antibodies or T-cell receptors.

29. A method according to claim 28, in which the library is constructed from nucleic acids isolated from an organism which has been challenged with an antigen.

30. A method according to any preceding claim, in which the entity is selected from a peptide, a polypeptide, a protein, a nascent polypeptide, an intracellular polypeptide precursor, a genomic DNA, a messenger RNA, a transfer RNA, a subcellular structure and an intracellular pathogen.

31. A method according to any preceding claim, in which the entity is associated with a predetermined condition of the cell or an organism from which the cell is derived.

32. A method according to claim 31, in which the condition is Alzheimer's Disease or Down's Syndrome, and the entity is selected from a neurofibrillary tangle, a senile plaque, a mutant beta amyloid precursor protein, a mutant ubiquitin-B protein, a frameshifted RNA encoding a mutant beta amyloid precursor protein, and a frameshifted RNA encoding a mutant ubiquitin-B protein.

33. A method according to claim 31, in which the condition is Creutzfeld-Jacob Disease (CJD), new variant CJD, or Bovine Spongiform Encephalopathy, and the entity is an infectious form of the prion protein (PrPSc).

34. A method according to claim 31 in which the condition is AIDS or an autoimmune disease.

35. A method according to any of claims 1 to 31, in which the entity is a mutant oncogenic protein.

36. A method according to claim 35, in which the mutant oncogenic protein is p21 ras.

37. A method according to claim 34 or 35, in which one of the first reporter and second reporter binds to a target present in the mutant oncogenic protein but not in a corresponding wild type protein, and the other of the first reporter and second reporter binds to a target present in both the mutant oncogenic protein and the wild-type protein.

38. A method according to any of claims 1 to 31 and 35, in which the entity is a chimaeric fusion protein resulting from a chromosomal translocation.

39. A method according to claim 38, in which the chimaeric fusion protein is a BCR-ABL fusion protein.

40. A method according to claim 38 or 39, in which one of the first reporter and the second reporter binds to a target comprising an SH2 domain, and the other of the first reporter and the second reporter binds to a target comprising an SH2-binding site.

41. A method according to any of claims 1 to 31 and 35, in which the entity is a mutant p53 protein.

42. A method according to claim 41, in which the p53 protein is a tetramer formed of p53 subunits.

43. A method according to claim 41 or 42, in which the first reporter and the second reporter bind to identical targets in the mutant p53 protein.

44. A method according to any preceding claim, in which one or both of the first and second reporters is provided as a fusion protein.

45. A method according to any of claims 1 to 43, in which one or both of the first and second reporters comprises two moieties linked by chemical coupling.

46. A method according to any preceding claim, in which the first reporter and the second reporter bind to different targets.

47. A method according to any of claims 1 to 45, in which the first reporter and the second reporter bind to the same target.

48. A pharmaceutical composition comprising an immunoglobulin-caspase fusion protein or conjugate, or a nucleic acid encoding an immunoglobulin-caspase fusion protein, together with a pharmaceutically acceptable carrier or diluent.

49. An immunoglobulin-caspase fusion protein or conjugate, or a nucleic acid encoding an immunoglobulin-caspase fusion protein for use in a method of treatment or diagnosis of a cancer in a human or animal.

50. Use of an immunoglobulin-caspase fusion protein or conjugate, or a nucleic acid encoding an immunoglobulin-caspase fusion protein for the preparation of a medicament for the treatment or diagnosis of cancer in a human or animal.

51. A method of destruction of a polypeptide in a cell, the method comprising the steps of:
(a) providing a cell comprising a polypeptide;
(b) providing a first reporter and a second reporter, in which a stable interaction of the first reporter with the second reporter leads to generation of protease activity; and
(c) allowing the first reporter and the second reporter to bind to the polypeptide, such that binding of the reporters to the polypeptide leads to stable interaction of the first reporter with the second reporter, generation of protease activity and proteolysis of the polypeptide.

52. A method of identifying the function of a gene, the method comprising the steps of:
(a) providing a cell comprising a gene encoding a polypeptide;
(b) providing a first reporter and a second reporter, in which a stable interaction of the first reporter with the second reporter leads to generation of protease activity;
(c) allowing the first reporter and the second reporter to bind to the polypeptide, such that binding of the reporters to the polypeptide leads to stable interaction of the first reporter with the second reporter, generation of protease activity and proteolysis of the polypeptide; and
(d) observing a phenotype.

53. A method according to any preceding claim, in which the signal is generation of a protease activity associated with a proteasome, preferably a 26S proteasome.

54. A method according to claim 53, in which the first reporter and the second reporter each comprise one or more domains of a F-box motif.

55. A method according to claim 53 or 54, in which the binding of the reporters to the entity leads to ubiquitination of the entity, or a polypeptide comprising the entity.

56. A method according to any of claims 53, 54 or 55, in which the binding of the reporters to the entity leads to proteolysis of the entity, or a polypeptide comprising the entity.
